(19) United States
(12) Patent Application Publication
(10) Pub. No.: US 2012/0076776 A1
(43) Pub. Date: Mar. 29, 2012

(54) METHOD FOR TREATING INFLAMATION BY LYMPHOCYTE DEPLETION OR SEQUESTERING

(76) Inventors: ERIC ELSTER, KENSINGTON, MD (US); DOUG TADAKI, FREDERICK, MD (US); JASON HAWKSWORTH, SILVER SPRING, MD (US); THOMAS DAVIS, OAK HILL, VA (US)

(21) Appl. No.: 13/021,451

(22) Filed: Feb. 4, 2011

Related U.S. Application Data
(60) Provisional application No. 61/301,383, filed on Feb. 4, 2010.

(51) Int. Cl. A61K 39/395; A61P 29/00; A61P 43/00; A61K 31/137

(52) U.S. Cl. 424/133.1; 424/184.1; 424/173.1

(57) ABSTRACT

A method for preventing inflammation, comprising administering to a subject a lymphocyte sequestrating or depletion agent before the onset of inflammation. A method for treating inflammation caused by an injury or an infection, comprising depleting immune lymphocyte of a subject by administering to said subject a lymphocyte sequestrating or depletion agent during or after said event. A method for preventing or treating abdominal adhesion comprising administering to a subject a lymphocyte sequestering or a lymphocyte depletion agent.

### CROSS-REFERENCE TO REALTED APPLICATIONS

This application claims priority to U.S. Provisional application 61/301,383, filed Feb. 4, 2010.

### BACKGROUND

This invention relates to a method for treating and preventing inflammation. More specifically, the invention is related to a method for treating or preventing ischemia injuries by depleting or sequestering lymphocytes prior to reperfusion.

Inflammation is part of the complex biological response to harmful stimuli, such as pathogens, damaged cells, or irritants. Inflammation is a protective attempt by mammal to remove the injurious stimuli and to initiate the healing process. Inflammation is typically caused by an infection or an injury. Without inflammation, wounds and infections would never heal. However, chronic inflammation can also lead to a host of diseases, such as hay fever, atherosclerosis, and rheumatoid arthritis.

Inflammation can be classified as either acute or chronic. Acute inflammation is the initial response of the body to harmful stimuli, and is achieved by the increased movement of plasma and leukocytes (especially granulocytes) from the blood into the injured tissues. A cascade of biochemical events propagates, and matures the inflammatory response, involving the local vascular system, the immune system, and various cells within the injured tissue. Prolonged inflammation, known as chronic inflammation, leads to a progressive shift in the type of cells present at the site of inflammation and is characterized by simultaneous destruction and healing of the tissue from the inflammatory process.

Mammalian immune system has evolved to respond to localized injury and infection. Such response is necessary for injury recovery and often has three main focuses: maintaining homeostasis, protection against microorganism invasion, and initiation of tissue repair. However, severe trauma and hemorrhagic shock induce a systemic activation of the immune system, which provokes the simultaneous activation of potent molecular and cellular components of innate immunity. These components include complements, cytokines, chemokines, neutrophils, and monocytes (21). Inappropriate inflammatory response often leads to secondary injuries to the host, which manifests clinically as systemic inflammatory response syndrome, acute respiratory distress syndrome, or even multiple-organ dysfunction syndrome. All of which are common posttraumatic complications found in intensive care units (1). Therefore, it is important to study therapeutic modulation of the immunologic response to injury in order to reduce trauma-associated morbidity and mortality.

Ischemia is a restriction in blood supply, generally due to factors in the blood vessels, with resultant damage or dysfunction of tissue. After an injury, ischemic reperfusion injury refers to tissue damage caused when blood supply returns to the tissue after a period of ischemia. The absence of oxygen and nutrients from blood creates a condition in which the restoration of circulation results in inflammation and oxidative damage through the induction of oxidative stress rather than restoration of normal function. The damage of reperfusion injury is due in part to the inflammatory response of damaged tissues. White blood cells, carried to the area by the newly returning blood, release a host of inflammatory factors such as interleukins as well as free radicals in response to tissue damage. The restored blood flow reintroduces oxygen within cells that damages cellular proteins, DNA, and the plasma membrane. Damage to the cell's membrane may in turn cause the release of more free radicals. Such reactive species may also act indirectly in redox signaling to turn on apoptosis. Leukocytes may also build up in small capillaries, obstructing them and leading to more ischemia.

Repeated bouts of ischemia and reperfusion injury also are thought to be a factor leading to the formation and failure to heal of chronic wounds such as pressure sores and diabetic foot ulcers (3). Continuous pressure limits blood supply and causes ischemia, and the inflammation occurs during reperfusion. As this process is repeated, it eventually damages tissue enough to cause a wound (3).

The majority of research on clinical therapeutics for ischemia reperfusion injury has been focused on monocyte and neutrophil adhesion blockade. However, despite promising preclinical data, results of phase 2 and 3 trials of neutrophil anti-adhesion therapy in ischemia-reperfusion disorders have been disappointing (2) In two clinical trials testing humanized CD18 monoclonal antibodies in the setting of traumatic injury, mortality and other primary end points were not significantly affected. (3, 4) These failures are likely the result of the redundancy of adhesion pathways, but also suggest that the neutrophil is not central in the innate immune response to ischemia-reperfusion injury (IRI).

Lymphocytes are major components of adaptive immunity and influence immune dysfunction following severe injury (5-7). Lymphocyte activation is classically described in the presence of foreign antigen bound to self-MHC molecules together with antigen-presenting cell costimulation signals. However, there is emerging evidence that lymphocytes are rapidly activated in an alloantigen-independent manner in the setting of ischemia reperfusion injury (IRI)(46). Danger signals released in IRI may activate lymphocytes and lead to innate function prior to classical adaptive function. (22) Furthermore, hypoxia may be sufficient for lymphocyte activation, as CD4+ T cells have been shown to increase adhesion to endothelial monolayers following anoxia modulation. (23, 24) Compelling pre-clinical investigation has established the innate role of lymphocytes in renal (8), gut (9), and liver (10, 11) IRI. In a renal IRI model, genetically engineered mice deficient in both CD4+ and CD8+ lymphocytes had substantially less kidney dysfunction after renal ischemia than did wild-type control mice (8). Interestingly, mice deficient in CD4+ and CD8+ lymphocytes demonstrated less tissue neutrophil infiltration, suggesting that lymphocytes orchestrate cell-mediated innate responses to ischemia. This was associated with a decrease in neutrophil infiltration, which was restored with adoptive transfer of wild type T cells into the athymic (nu/nu) mice.

Therefore, immunomodulation of lymphocytes may offer a novel approach to attenuate detrimental immune responses to inflammation. For example, depletion or sequestering lymphocytes may ameliorate secondary morbidity and mortality associated with severe hemorrhage.

Natural killer (NK) cells are part of the innate immune system. They are thought to play an important role in the development of such syndromes by interplay with other immune cell types and subsequent activation of the inflammatory cascade. In a reported study, NK cells were depleted by administration of antimouse asialo-GM1 antibody in a murine polytrauma model consisting of femur fracture, hemorrhagic shock, and subsequent sepsis. Mortality and immune parameters such as cytokine expression in lung and liver, lymphocyte phenotyping, lymphocyte apoptosis, and organ pathology were determined 96 hours after sepsis induction. NK cell depletion resulted in 50% mortality reduction. Furthermore, reductions in the inflammatory response were observed, represented by IL-6 expression in liver, and a decrease in infiltrating neutrophils in the liver and lung. In addition, lymphocyte apoptosis in spleen was decreased by depletion of NK cells. Taken together, these data demonstrate that NK cells contribute to the pathogenetic pathways in a murine polytrauma model (43). T cell and B cell lymphocytes have not been studied as to their roles in preventing or reducing secondary immunologic injury in surgical and trauma patients following hemorrhagic shock.

Adhesions are fibrous tissues that form between intra-abdominal organs following abdominal and pelvic surgery (56). They are common, occurring following 68 to 100% of all such operations. They result from the peritoneal response to mechanical injury, intra-abdominal ischemia, and the presence of foreign material (e.g. synthetic mesh used for ventral hernia repair) within the abdomen (57). The clinical implications of this wound healing process are profound; and common sequelae including intestinal obstruction, chronic abdominal pain, infertility, pelvic pain, and increased complications during subsequent surgical procedures. Indeed, adhesions are the most common cause of intestinal obstruction in the Western world, accounting for 1% of all hospital admissions and resulting in morbidity costing an estimated $1-3 billion annually in the United States alone (58-60).

Patients that have had prior bowel obstruction requiring surgical correction are at increased risk for subsequent adhesive obstructions (60, 61). Specifically, the recurrence rate for patients requiring a single exploration for adhesive small bowel obstruction is 18-50% within 10 years. The relative risk of the recurrent obstruction increases with increased number of prior episodes, reaching 81% for patients with four or more admissions for adhesive obstruction. Various types of adhesions (e.g. matted and pelvic) can greatly increase the risk of recurrence and 58% of recurrences occur within the first 5 years following therapy (61).

There is currently no effective preventative therapy for adhesions. Surgical adhesiolysis is therapeutic but results in recurrent adhesive disease and markedly increases the risk of subsequent adhesion related complications. Recent small animal models have demonstrated that T lymphocytes are required for the formation of the abdominal adhesions and their sequelae (62). Clinically, it has been empirically observed that patients undergoing extensive abdominal procedures for pancreatic transplantation do not form adhesions, particularly following therapeutic T cell depleting antirejection therapy.

Many attempts have been made in limiting adhesion formation, one of which was placing mechanical barriers between the intra-abdominal organs and the damaged peritoneum. This procedure has been demonstrated to have limited efficacy particularly in preventing inter-loop and pelvic adhesions (63, 64). The inability of barrier agents to eliminate surgical adhesions stems from failure to arrest the underlying problem: that of dysregulated fibrogenesis during wound healing. Specifically they do not alter the activity of T lymphocytes, the key regulator of adhesion formation.

Control of pathological T cell responses has been extensively studied and reduced to routine practice in the field of solid organ transplantation. T cell depletion and T cell specific immunosuppression is routinely used in patients undergoing major abdominal operations such as pancreas and liver transplantation. Specific T cell depleting agents included polyclonal rabbit anti-thymocyte globulin (Thymoglobulin, Genzyme, Cambridge, Mass.) which has antibodies directed against many antigens on lymphocytes. Treatment with Thymoglobulin has been shown to result in a profound reduction in the number of T lymphocytes at the time of transplantation, therefore decreasing the frequency of alloreactive cells at a time of increased immune susceptibility. In both kidney and pancreas transplant recipients this has been achieved without an increase in postoperative infections or impaired wound healing relative to transplant patients without T cell depletion, essential elements for the broader application of the prevention of surgical adhesions (65, 66). Therefore, depletion or sequestration of lymphocytes at the time of operation, combined with modest inhibition of Th1 type T cell activation during the perioperative period, may prevent adhesion formation, and therefore eliminate subsequent complications of recurrent adhesive disease.

Antithymocyte globulin (ATG) is a potent lymphocyte depleting agent that is used clinically in induction and anti-rejection therapy for solid organ transplantation, treatment of graft versus host disease, and selected autoimmune diseases. (12, 13) The immunosuppressive activity of anti-lymphocyte sera was first noted at the turn of the century when Metchnikoff described its anti-inflammatory properties. (14) The primary mechanism of immunosuppression involves massive peripheral and central lymphocyte depletion primarily by complement and Fas/Fas-L mediated apoptosis pathways. [15-17] In addition, ATG results in antibody inhibition of nondepleted T cells and functional alteration of several membrane receptors (TCR/CD3) and coreceptors (CD2, CD4, and CD8). [15, 18] Given the innate role of the lymphocyte in IRI, ATG may effectively modulate the post-traumatic inflammatory response.

While ATG is primarily used clinically to suppress adaptive lymphocyte responses, pre-clinical studies have demonstrated the utility of ATG in abrogating innate immune responses. In a non-human primate model, polyclonal ATG conferred a protective effect on reperfusion injury following limb ischemia. (27) In clinical kidney and liver transplantation, ATG (Thymoglobulin) has been shown to reduce graft dysfunction associated with IRI. (28) Specifically, ATG has been shown to reduce delayed graft function following transplantation, an event that is thought to be related to IRI.

FTY720 (NOVARTIS®) is an immunomodulator currently in phase III clinical trials that sequestrates lymphocytes to secondary lymphoid organs and reduces circulatory lymphocytes by targeting receptors for sphingosine 1-phosphate. [49] FTY720 has been shown to ameliorate IRI in various animal models and lymphocyte modulation has been mechanistically implicated. [50] In a rat renal transplant model, FTY720 treatment improved renal function and reduced intragraft neutrophil infiltration despite no change in adhesion molecules expression, highlighting the central role of the lymphocyte in IRI. [51]

The innate role of lymphocytes offers the potential for preventing or treating inflammation, especially those associated with ischemic reperfusion injury, abdominal adhesion or infection. Immumodulation via disruption of lymphocyte trafficking with FTY720 or depletion of lymphocytes is a viable therapeutic strategy for such treatment option.

### DESCRIPTION OF THE FIGURES

FIG. 1. A) Schematic illustrating experimental design for control group. B) Schematic illustrating experimental design for PATG group.

FIG. 2. Hemodynamic profiles during hemorrhage period for experimental and control groups. A) Heart rate. B) Mean arterial pressure. C) Cardiac output.

FIG. 3. Kaplan-Meier survival curve for experimental groups versus control with log-rank test for statistical comparison. A) Overall survival. B) Reperfusion period survival.

FIG. 4. Peripheral lymphocyte counts during hemorrhage and reperfusion periods (using PATG). One-way ANOVA with repeated measures design was used for statistical comparison. Error bars represent ±SEM.

FIG. 5. Representative FACS plots for CD3+CD4+ and CD3+CD8+ peripheral lymphocytes. A) Sample lymphocyte gating and IgG plots for control and PATG groups. B) CD3+CD4+ and CD3+CD8+ peripheral lymphocytes at time of liver injury (t=0). C) CD3+CD4+ and CD3+CD8+ peripheral lymphocytes at peak reperfusion period (t=24 hours). D) Quantification of CD3+CD4+ and E) CD3+CD8+ lymphocytes throughout the experiment. One-way ANOVA with repeated measures design was used for statistical comparison. Error bars represent ±SEM.

FIG. 6. Central lymphocyte counts at time of necropsy. Quantification of CD3+ reactivity by ACIS. * indicates p<0.05 compared to normal, unmanipulated tissue. Data is depicted as mean ±SEM.

FIG. 7. Peripheral neutrophil counts during hemorrhage and reperfusion periods. One-way ANOVA with repeated measures design was used for statistical comparison. Error bars represent ±SEM.

FIG. 8. Lung tissue neutrophil counts at time of necropsy. B) Quatification of MPO+ reactivity. * indicates p<0.05 compared to normal, unmanipulated tissue. † indicates p<0.05 compared to control group. Data is depicted as mean ±SEM.

FIG. 9. Gene transcript expression in liver tissue of PATG relative to the control group. Median value of relative fold expression is depicted on a logarithmic scale. >2 fold difference from control was considered statistically significant and is indicated by *.

FIG. 10. Experimental design. Liver injury was initiated at t=0. Uncontrolled hemorrhage occurred until 1 hour, at which time the abdomen was packed and temporarily closed (pre-hospital phase). The animal was observed until 2 hours when hospital care was initiated. The liver was repaired and the abdomen definitively closed. The animal was then observed for a total of 72 hours. Blood transfusion was administered as indicated. Necropsy was performed when the animal expired or at 72 hours following euthanasia. The uncontrolled hemorrhage and pre-hospital phases were considered the hemorrhage period, while the hospital care phase was considered the reperfusion period. In the experimental group, FTY720 (0.3 mg/kg) was administered 15 minutes following liver injury.

FIG. 11. Hemodynamic profiles during hemorrhage period for experimental and control groups. a) Heart rate. b) Mean arterial pressure. c) Cardiac output.

FIG. 12. Kaplan-Meier survival curve for experimental groups versus control with log-rank test for statistical comparison. a) Overall survival. b) Reperfusion period survival. Central lymphocyte counts at time of necropsy. Dashed red line indicates end of hemorrhage and beginning of reperfusion period. C) Quantification of CD3+ reactivity by ALIS. * indicates p<0.05 compared to normal, unmanipulated tissue; † indicates p<0.05 compared to control. Data is depicted as mean ±SEM.

FIG. 13. Peripheral neutrophil counts during hemorrhage and reperfusion periods. b) Quatification of MPO+ reactivity. * indicates p<0.05 compared to normal, unmanipulated tissue. † indicates p<0.05 compared to control group. Data is depicted as mean ±SEM. c) Inflammatory gene transcript expression in liver tissue of PATG relative to the control group. Mean value of relative fold expression is depicted on a logarithmic scale.

FIG. 14. Dosing Schema for Thymoglobulin

### DETAILED DESCRIPTION OF THE INVENTION

It is shown in this application, lymphocyte (especially T or B lymphocytes) sequestration or depletion significantly prevents or treats inflammation caused by an infection or injury, such as ischemic reperfusion injury caused by a trauma or abdominal adhesion after an surgery. Lymphocyte immunomodulation appears to attenuate innate cellular and molecular activation. Specifically, disruption of the innate lymphocyte response resulted in significantly decreased circulating and lung tissue infiltrating neutrophils and decreased expression of inflammatory genes.

To prevent a harmful inflammation, before the onset of an inflammation, the immune lymphocytes of a subject can be modulated (depleted or sequestered) by administering to the subject one or more doses of a lymphocyte depletion or a sequester agent or both. For example, one or more doses of a lymphocyte depleting or sequestering agent may be administered to a subject during or after an inflammation causing event, such as an injury or an infection.

The lymphocyte modulated may include B lymphocytes, T lymphocytes, NK cells, and platelets. An example of a lymphocyte sequestering agent is FTY720. The lymphocyte depletion agent may include PATG, Thymoglobulin (Genzyme, Cambridge, Mass.) or Alemtuzumab (Genzyme, Cambridge, Mass.). The lymphocyte depletion/sequestering agent may be administered within 7 days of an inflammation via a variety of routes, including oral, transdermal, transmucosal, intrademal, subcutaneous, intravenous and intramuscular routes. The agent may be administered with a pharmaceutical carrier. Alternatively, one or more doses of a lymphocyte depleting or sequestering agent may be administered to a subject during or after an inflammation causing event, such as an injury or an infection. In particular, administering one or more doses of a lymphocyte depletion or a sequestering agent before, during or after hemorrhagic shock can treat ischemic injuries associated with hemorrhagic shock and improves survival.

To treat a harmful inflammation, upon the onset of an inflammation, the immune lymphocytes of a subject is depleted or sequestered by administering to the subject one or more doses of a lymphocyte depletion or a sequester agent or both. An example of a lymphocyte sequestering agent is FTY720. The lymphocyte depletion agent may include PATG, Thymoglobulin (Genzyme, Cambridge, Mass.) or Alemtuzumab (Genzyme, Cambridge, Mass.). The lymphocyte depletion/sequestering agent is administered during the inflammation via a variety of routes, including oral, transdermal, transmucosal, intrademal, subcutaneous, intravenous and intramuscular routes. The agent may be administered with a pharmaceutical carrier.

A method for preventing or treating abdominal adhesion may comprise depleting or sequestering immune lymphocytes. Such depletion or sequestration may be accomplished by administering to a subject one or more doses of a lymphocyte depletion agent or a lymphocyte sequestering agent or both. The lymphocyte depletion agent may include PATG, Thymoglobulin and Alemtuzumab. The lymphocyte sequestering agent may be FTY720. The administration may be carried out via oral, transdermal, transmucosal, intrademal, subcutaneous, intravenous, or intramuscular routes. The agent may be administered with a pharmaceutically acceptable carrier. The agent may be administered to the subject before, or after the injury such as a surgery.

### EXAMPLE 1

### Lymphocyte Depletion using PATG on Experimental Hemorrhagic Shock Induced by Porcine Liver Injury

### Development of Porcine Anti-Thymocyte Globulin (PATG)

Porcine thymuses were obtained aseptically from normal unmanipulated control pigs, and single-cell suspensions were prepared by gentle pressing thorough a nylon filter mesh (Tetko, Inc, Elmsford, N.Y. USA) into cold RPMI 1640 medium (GIBCO, Grand Island, N.Y. USA). Thymocytes were isolated on density gradients using Ficoll-Paque (Pharmacia Biotech, Uppsala, Sweden) to remove reds cells and granulocytes, washed three times and then resuspended in Dulbecco phosphate buffered saline (D-PBS; GIBCO). All cell preparations had a purity of >95%.

To produce polyclonal antibody against porcine thymocytes, 125 adult female New Zealand White rabbits (Covance, Denver, Pa.) were immunized subcutaneously with 5×10⁶ purified porcine thymocytes in complete Freund's adjuvant (Sigma, St. Louis, Mo., USA). Subsequent intravenous booster immunizations of 5×10⁶ thymocyteswere given on days 14, 28 and 42. The rabbits were terminally bled, by terminal cardiac puncture, 7 days after the final immunization. The collected blood was pooled and sera isolated by centrifugation. The Immunoglobulin fraction was purified using Protein G Sepharose-4 Fast Flow columns (Pharmacia), sterile filtered (0.2 μm) and stored at 4° C. before use. All PATG used in these studies was obtained from a single batch and was tested to be endotoxin-low (<0.05 IU/mg) by QCL-1000 Chromogenic LAL (Cambrex BioWhitaker Biosciences, Walkersville, Md.).

The titer of antiserum was tested by FACS analysis to measure antibody binding-coating of PATG to the cell surface of purified porcine thymocytes and peripheral blood leukocytes (gated lymphocyte, monocyte and granulocyte cell populations). Cells (1×10⁶/100 μl) were incubated at 4° C. with PATG at concentrations ranging from 0.01 μg/ml to 100 μg/ml, washed, and incubated with FITC-labeled goat anti-rabbit antibody (Jackson ImmunoResearch Laboratories, West Grove, Pa.). Results were compared to with cells incubated with the same concentrations of unspecific rabbit Ig. The PATG exhibited stronger titer to thymocytes and peripheral blood CD4⁺ T-cells (receptor sites saturated ≧0.5 μg/1×10⁶ cells) than to peripheral blood granulocytes and monocytes (100 to 300-fold greater; data not shown). A dose-response experiment was performed, and determined that 4 PATG doses of 10 mg/kg of weight) 24 hours apart were necessary for >50% sustained lymphocyte depletion.

### Animal Preparation

Male and female 3-12 month Yorkshire (Sus scrofa domestica) swine weighing 25-35 kg were acquired from ABI farms (Donsboro, Pa.). Feed was withheld 12 hours before surgery. Prior to surgery, animals were sedated and anesthesia induced with intramuscular ketamine hydrochloride (12-20 mg/kg of weight) and xylazine (2.2 mg/kg of weight) as well as atropine sulfate (0.05 mg/kg of weight) to decrease tracheal secretions. Mask ventilation with Isoflurane (5.0%) was used to facilitate endotracheal intubation. Pigs were ventilated (Ohmeda 7800 series ventilator, Datex, Madison, Wis.) at 12-15 breaths/min and tidal volume 10 mL/kg. Anesthesia was maintained with Isoflurane (1.5-2.5%) in 21-25% O2.

Following adequate anesthesia the right external and internal jugular veins and carotid artery were isolated. A 9Fr dual-lumen, tunneled Hickman catheter was placed in the external jugular vein for drug infusion and blood collection during the survival period. A 9Fr introducer sheath was placed in the internal jugular vein and a 7.5Fr pulmonary artery catheter (PAC; Edwards Life Sciences, Irvine, Calif.) was inserted for continuous hemodynamic monitoring. An 18 G Angiocath was placed in the carotid artery and mean arterial pressure (MAP) was continuously transduced. A midline laparotomy was performed to expose the liver and isolate the left lateral lobe. Urine was collected via bladder catheterization. Rectal temperature was monitored continuously. Normothermia (37° C.) was maintained with a warming device (Model 505, Bair Hugger, Augustine Medical, Eden Prairie, Minn.).

### Liver Injury and Resuscitation

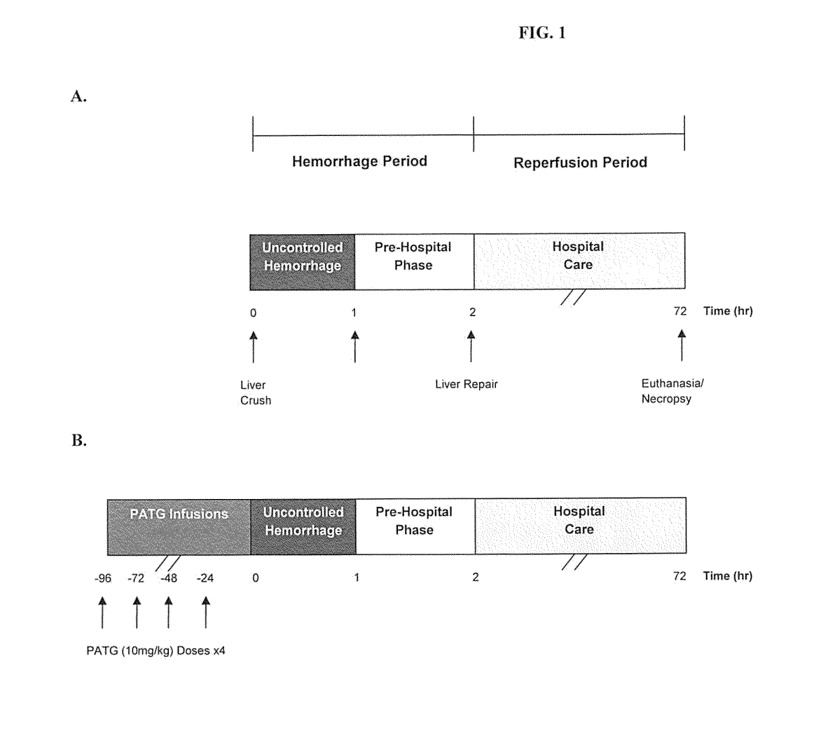

All animal groups underwent a standardized liver injury and resuscitation protocol. (FIG. 1A) A reproducible liver injury was created by placing a ring clamp over the left lower lobe, 50% in width and 3.5-5 mm from the apex, adjusting for the relative size of the animal. (19) The clamp was closed and a 10 blade was used to lacerate the lobe from the top of the clamp through the remaining width. The liver injury denoted the start of the pre-hospital phase (Time 0). After 1 minute, the clamp was removed and the remaining tissue excised, resulting in a 25% lobectomy, consistent with a grade III liver injury. The hepatectomy specimen was weighed and divided by animal weight (kg) and reported as hepatectomy weight index (g/kg). Bleeding was spontaneous and removed continually via suction and quantified by weight. Blood loss weight (g) was used to calculate estimated blood loss volume (%). At 15 minutes, animals were infused 250 mL NS over the next 60 minutes. This time point represents the arrival of a first responder (e.g. medic) who has limited carrying capacity. After 1 hour blood collection was discontinued and the abdomen packed then closed with towel clips.

After a total of 2 hours, the pre-hospital phase was completed and hospital arrival was simulated. This time point represents arrival of the patient to a hospital with surgical capabilities. The abdomen was reopened, residual blood suctioned, sponges collected, and blood loss quantified by weight. The liver injury was repaired with suture and the abdomen definitively closed. Invasive lines were removed and the carotid artery repaired. One unit of allogeneic whole blood (10 mL/kg) (Thomas Morris, Reisterstown, Md.) was administered for hemoglobin <7 g/dL, otherwise animals received additional NS at 10 mL/kg. Anesthesia was stopped and the animals extubated.

Animals in the PATG group were anesthetized and underwent tunneled Hickman catheter placement 4 days prior to liver injury. PATG (10 mg/kg of body weight) was diluted in 250 mL NS and infused daily for 4 doses total (FIG. 1B). Several animals experienced hemodynamic instability during the first infusion of PATG, likely secondary to cytokine release phenomena. Subsequently, the protocol was modified and one dose of Solumedrol (2 mg/kg) was administered prior to only the first PATG infusion which abrogated this drug effect.

### Post-Surgical Care

Pigs were allowed to eat a regular diet and drink water following surgery. Buprenorphine (0.05-0.1 mg/kg IM/IV) was administered every 6-12 hours if the animals demonstrated any sign of pain. If the pigs showed any sign of severe disability (e.g. inability to ambulate, eat, drink), severe infection, or uncontrolled pain, they were euthanized (EUTHASOL™, Virbac AH, Fort Worth, Tex.) and taken for necropsy. At the end of the survival period (72 hours post-injury), surviving pigs were euthanized and taken for necropsy. Samples of the heart, lung, liver, kidney, small intestine, mesenteric lymph node, and spleen were taken. A portion of each sample was submitted for histology. Additional samples were placed in RNALater (QIAGEN®, Valencia, Calif.) and flash frozen using liquid nitrogen within 48 hours. These samples were stored at −80° C.

### Laboratory Data

All functional laboratory assays were performed at 37° C., consistent with recorded normothermic animal temperatures (37.5±0.9° C.). Blood samples were collected at 0, 15, 30, 60, 90, and 120 minutes during the hemorrhage period and at 18, 24, 36, 48, and 72 hours during the survival period. Complete blood count with differentiation was performed with a cell counter (NDvia120 Hematology System, Siemens, Deerfield, Ill.).

### Flow Cytometry

Whole blood was fractionated using Histopaque 1077 (Sigma, St. Louis, Mo.) density gradient centrifugation. Fluorescent immunostaining of the isolated mononuclear cells was accomplished via a 30-minute incubation at 4° C. with fluorescein isothiocyanate (FITC)-labeled CD3 and either phycoerythrin (PE)-labeled CD4 or CD8 mouse anti-porcine antibodies (BD Pharmingen, San Jose, Calif.). The cells were washed in FACS buffer (3% FBS, 1% sodium azide), fixed using 1.6% paraformaldehyde, and quantified by flow cytometry (Beckman-Coulter, Hialeah, Fla.). Appropriate mouse IgG isotype controls were used (BD Pharmingen, San Jose, Calif.). Flow cytometry analysis was performed with FACS Calibur (Becton Dickinson). Statistical analysis of the FACS data was performed with CellQuest (Becton Dickson).

### Immunohistochemistry

Tissue slides were deparaffinized using xylene followed by graded baths of ethanol. A DAKO Autostainer Plus Universal Staining System (DAKO, Carpenteria, Calif.) was used for automated immunohistochemical staining.

Immunohistochemical detection of CD3 was performed on sections of formalin fixed, paraffin embedded blocks of swine lymph node and spleen. Antigen retrieval was performed using Trilogy (Cell Marque, Rocklin, Calif.) for 30 minutes. Rat, anti-human CD3, monoclonal antibody (AbD Serotec, Raleigh, N.C.) was used at a dilution of 1:50 and incubated overnight at 4 C. Biotinylated goat anti-rat IgG specific polyclonal antibody (BD Biosciences, San Jose, Calif.) was applied as a secondary antibody at a 1:100 dilution for 30 minutes at room temperature. The chromogen applied was 3, 3′ Diaminobenzidine (DAKO, Carpenteria, Calif.) for 10 minutes. The sections were counterstained with Hematoxylin (DAKO, Carpenteria, Calif.). External negative controls were processed identically as CD3 but the primary antibody was substituted with normal rat serum. A tissue sample was considered positive if reactive cells, lymphocytes, demonstrated reactivity. An Automated Cellular Imaging System (ACIS) was used to quantify the CD3 immunohistochemistry staining. By using the ACIS system, CD3 immunohistochemical staining could be specifically quantitated by determining how much brown (positive CD3 immunohistochemical reaction) was in the image compared to the amount of blue (hematoxylin nuclear counterstain).

Immunohistochemical detection of MPO was performed on sections of formalin fixed, paraffin embedded blocks of swine lung. Antigen retrieval was performed using Proteinase K (DAKO, Carpenteria Calif.) for 30 minutes. Polyclonal rabbit, anti-human MPO (DAKO, Carpenteria Calif.) was used at a ready to use dilution and incubated at room temperature for 30 minutes. Envision, anti-rabbit (DAKO, Carpenteria Calif.) was applied as a secondary antibody for 30 minutes at room temperature. The chromogen applied was 3,3′ Diaminobenzidine (DAKO, Carpenteria, Calif.) for 10 minutes. The sections were counterstained with Hematoxylin (DAKO, Carpenteria, Calif.). External negative control was processed identically as with MPO but the primary antibody was substituted with normal rabbit serum. A tissue sample was considered positive if reactive cells, neutrophils, demonstrated reactivity in conjunction with a segmented nucleus. Neutrophils were noted mainly in circulation in small septal vessels and in large vessels. Cells with appropriate nuclear morphology and the presence of light to intensely reactive granules were counted; 5 random 40× fields were assessed.

### RNA Extraction and Quantitative Real-Time Polymerase Chain Reaction

Total RNA was isolated from liver tissue taken at necropsy using Qiagen RNeasy Mini Kit (QIAGEN® Inc. Valencia, Calif.) according to manufacturer's instructions. RNA purity and quantity were assessed by measuring the A₂₆₀, A₂₈₀, and A₂₃₀ on a Nanodrop Spectrophotometer (NanoDrop Technologies Inc. Wilmington, Del.). RNA quality was determined from the 28S/18S rRNA ratio and RNA Integrity Number (RIN) using an Agilent 2100 BioAnalyzer (Agilent Technologies Inc. Santa Clara, Calif.). RIN values for all specimens in this study were >6.5. Reverse transcriptions were performed using Roche 1^{st} Strand Synthesis kits (Roche Diagnostics Corporation, Indianapolis, Ind.) according to the manufacturer's protocol. Quantitative real-time polymerase chain reaction (QRT-PCR) was performed using the 7900HT Fast Real-Time PCR System (Applied Biosystems, Foster City, Calif.) to assess mRNA transcript expression of 21 immune-related genes. Taqman chemistry was used. Primers for 18S rRNA target were used as an internal control for each reaction. Primers and probes for the targets of interest were obtained from Applied Biosystems (Table 1). All samples were run in duplicate. Individual samples were compared to averaged control tissue expression. Transcript quantification was derived using the comparative threshold cycle method (20) and reported as the median n-fold difference of the experimental sample to the control pool.

TABLE 1
RT-PCR targets with primer and probe sequences

| Target | Forward Primer | Reverse Primer | Probe |
|---|---|---|---|
| IL-1α | CTGAAGAAGAGAC | AAGTTGTATTTCAT | CAGAAGAAGAAATC |
| | GGTTGAGTTTAAA | GTTGCTCTGGAA | ATCAAGCCCAGATC |
| | | | AGC |
| IL-1β | TTGAATTCGAGTC | CCCAGGAAGACGGG | CAACTGGTACATCA |
| | TGCCCTGT | CTTT | GCACCTCTCAAGCA |
| | | | GAA |
| IL-6 | AATGTCGAGGCTG | TGGTGGCTTTGTCT | AGCACTGATCCAGA |
| | TGCAGATT | GGATTCT | CCCTGAGGCAAA |
| TNFα | TGGCCCCTTGAGC | CGGGCTTATCTGAG | CCCTCTGGCCCAAG |
| | ATCA | GTTTGAGA | GACTCAGATCA |
| EDN-1 | CCTGCTCTTCCCT | GGACAATGTGTTCT | AGTGTGTCTACTTC |
| | GATGGATAA | GGAGTGTTG | TGCCACCTGGACAT |
| | | | CA |
| INOS | CGTTATGCCACCA | AGACCCGGAAGTCG | ATCAGGTCGGCCAT |
| | ACAATGG | TGCTT | CACCGTG |
| HIF-1α | GCATTAAAGTTAG | AAGGACTAGCTGGC | CTGGAACTTTCTTT |
| | AACCAAATCCAGA | TGATCTTGA | TACCATGCCCCAGA |
| | GT | | TT |
| HSP70 | ACCATCGAGGAGG | ATGAAGTGATACAG | CCTCTTTGAGACCC |
| | TGGATTAGA | AACAGAACTAAAAT | AAGGACTTTGACAT |
| | | ACAG | TGT |
| TGF-β1 | AGGGCTACCATGC | CGGGTTGTGCTGGT | CCTAGACACTCAGT |
| | CAATTTCT | TGTACA | ACAGCAAGGTCCTG |
| | | | GC |
| C3 | CAAGAAATGATTG | GACCTGTGGTTCAC | AGCCTTTGTTCTCA |
| | GTGGCTTCAA | AGATGTCTTT | TCGCGCTGCA |
| TGFα | CCCAGATTCCCAC | AGCGTGCGCCGACG | AAGCCAGCATGTGT |
| | AGTCAGTTC | TA | CTGCCACTCTG |
| VCAM | CTGTTCAAGTTGC | GAGCAGGTCATGTT | CGACCATCTCCGTC |
| | TCCCAGGGAT | CACAGAACTG | AACCCCTCC |
| IL-2 | ACAGTTGCTTTTG | CCTGCTTGGGCATG | CGAGAATGCTGATC |
| | AAGGAAGTTAAGA | TAAAATTT | TCTCCAGGATGCTC |
| | A | | |
| CD154 | GAAATGCACAAAG | TGCTGTTTTACTAC | ATCCTCAAATTGCG |
| | GCGATCA | TGGCCTCG | GCACATGTCATAAG |
| BAX | CTCAAGCGCATTG | GTCCACGGCTGCGA | TCCTCTGCAGCTCC |
| | GAGATGA | TCA | ATGTTACTGTCCAG |
| BCL-2 | TTCTACTTTGTCA | TGTCCTTTGTCCCA | CAGGTCTTTATTGC |
| | GGCTTATGAAGGT | TAATAATTTTTTT | TACTCAATTTGAAT |
| | T | | CTTCCCA |
| FAS | AATGGTATCGAAG | CGGAGCAGCTGGAC | CATGCATGACAATC |
| | AAACCAAAATAGA | TTTCTG | CCAAAGATACAGCT |
| | C | | GA |
| FAS-L | GGCAAGCCTAACT | CATATACTTCACCC | CAATTCCATAGGTG |
| | CAAGATCCA | CAGAGACCAA | TCTTCCCATTCCAG |
| | | | AGG |
| NF-κB | CTGGCAGCTCTCC | CACGAGTCATCCAG | CTCAAAGTTCTCCA |
| | TCAAAGC | GTCATACAG | CCAGGGGATCTGCT |
| | | | C |
| COX-2 | CTGAACACCTCCG | AAGCACATCGCACA | CAGACCAGGCACCA |
| | CTTTGC | CTCTATTATGT | GACCAAAGACCTC |
| PPLA-2 | CTACTGTGGCCTA | TCTCTGTAGCAGTT | CGCAGCACCTGTCC |
| | GGTGGATCAG | GTCGTGTGTCT | AGTTCATCCAC |

### Statistical Analysis

Animals were randomly assigned to the control or experimental groups. Survival analysis between groups was performed by Kaplan Meier survival plots with the log rank test. Continuous data was compared with the Student t-test or nonparametric tests as appropriate. Multiple comparisons were performed with one-way ANOVA. A one-way ANOVA with repeated measures design was used for continuous time-dependent comparisons. Statistical analysis was performed using SPSS (SPSS Inc., Chicago, Ill.). A two-tailed p value <0.05 was considered statistically significant. All data is represented as means ±standard error (SEM) unless otherwise specified.

### Results

Animal Characteristics and Hemodynamic Profiles during Hemorrhage Period

Mean animal weight and gender distribution were not significantly different between the control (n=9) and PATG (n=8) groups (Table 2). The mean hepatectomy weight index was similar between groups indicating that liver injury was well standardized. In addition, the majority of animals in both groups required blood transfusion during the hospital phase for hemoglobin value less than 7 g/dL.

TABLE 2
Animal Characteristics

| Variable | Control (n = 9) | PATG (n = 8) | p-value |
|---|---|---|---|
| Mean animal weight [kg] | 32.5 ± 1.7 | 28.51 ± 0.8 | NS¹ |
| Animal gender-M:F | 5:4 | 4:4 | NS² |
| Mean hepatectomy weight index | 0.31 ± 0.04 | 0.33 ± 0.03 | NS¹ |
| [g/kg] | | | |
| Mean blood loss volume % | 46.8 ± 3.4 | 47.4 ± 4.0 | NS¹ |
| Transfusion requirement-no. (%) | 7 (78) | 5 (63) | NS² |

¹Student t-test;
²chi-squared

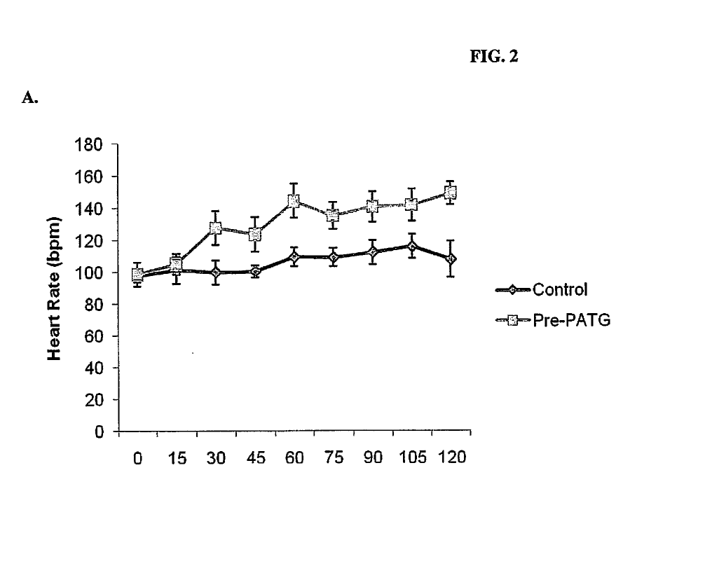

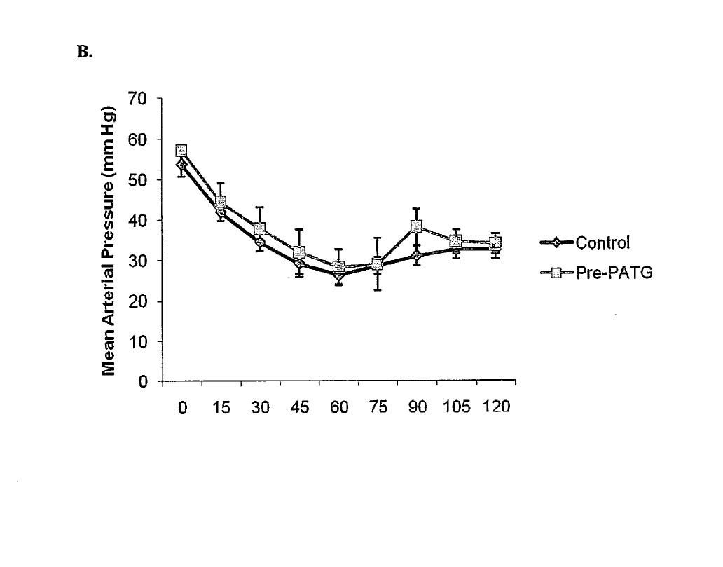

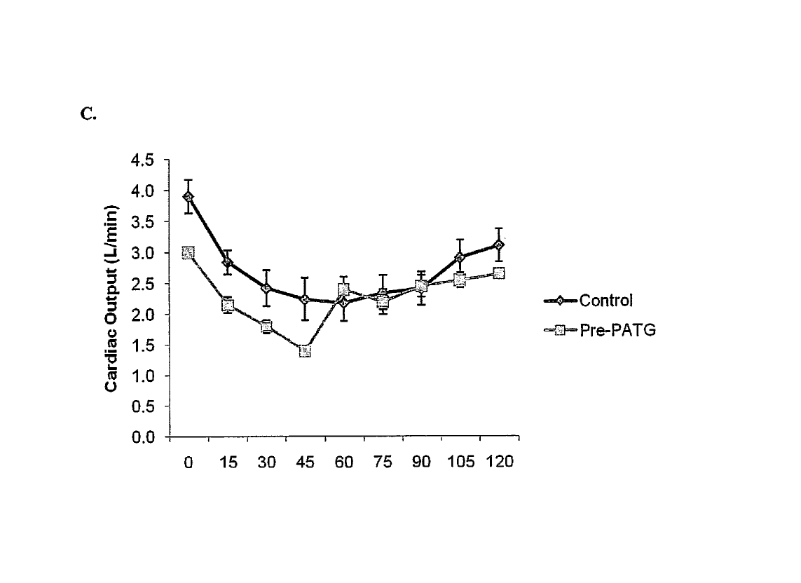

Invasive hemodynamic monitoring during the hemorrhage period demonstrated shock physiology in all animal groups (FIG. 2). Cardiac output and mean arterial pressure were significantly decreased and heart rate significantly increased compared to baseline. The heart rate increased markedly in the PATG group, possible due to cytokine release phenomena.

### Survival Analysis

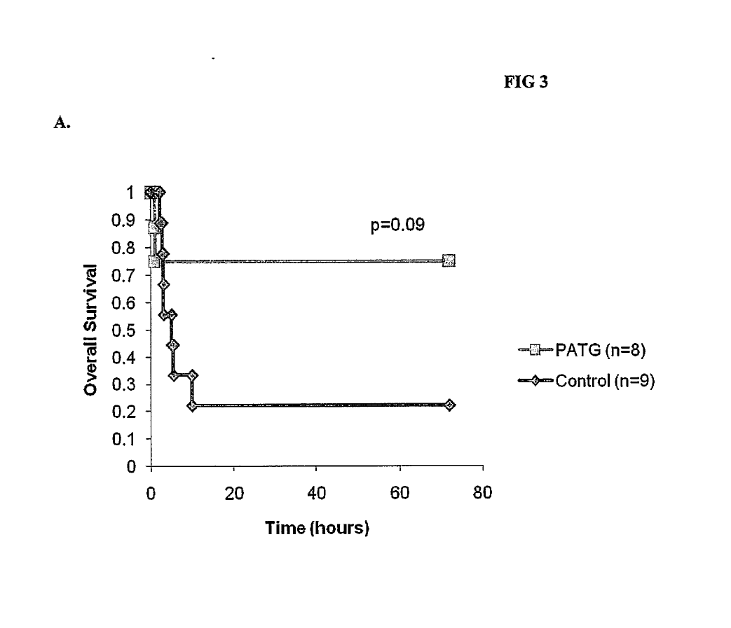

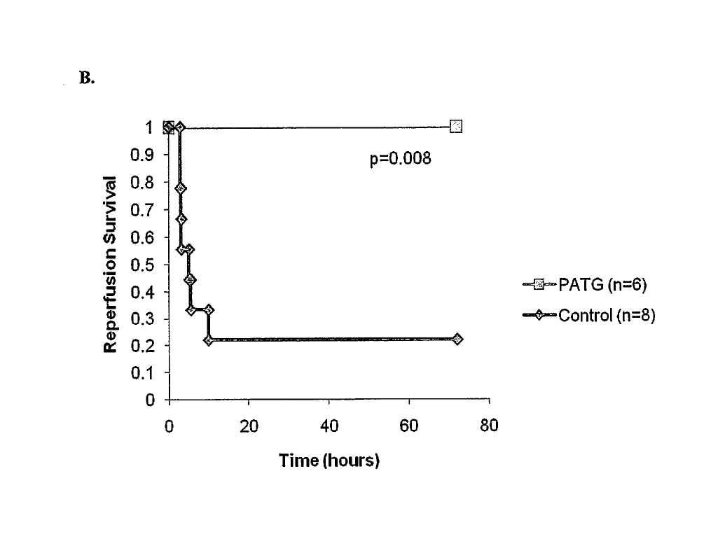

Overall survival in the experimental group was improved compared to control, although not statistically significant by log rank test (FIG. 3A). The control group survival was 22% (2/9) and PATG was 75% (6/8) p=0.09. During the reperfusion period (following hemorrhage) survival was statistically improved in the experimental group compared to control (FIG. 3B). Control group survival was 20% (2/8) and PATG was 100% (6/6) p=0.008 during reperfusion. Thereby, disruption of the innate lymphocyte response to hemorrhagic shock appeared to significantly reduce mortality associated with reperfusion injury.

### Peripheral and Central Lymphocyte Response to Hemorrhagic Shock

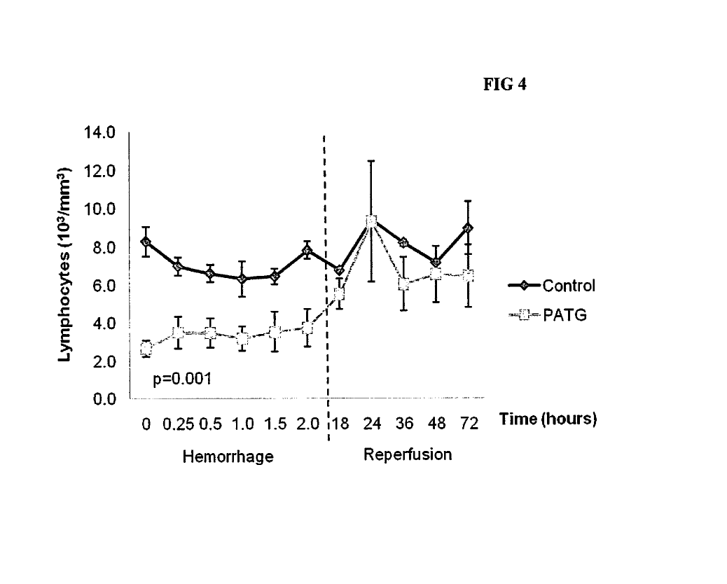

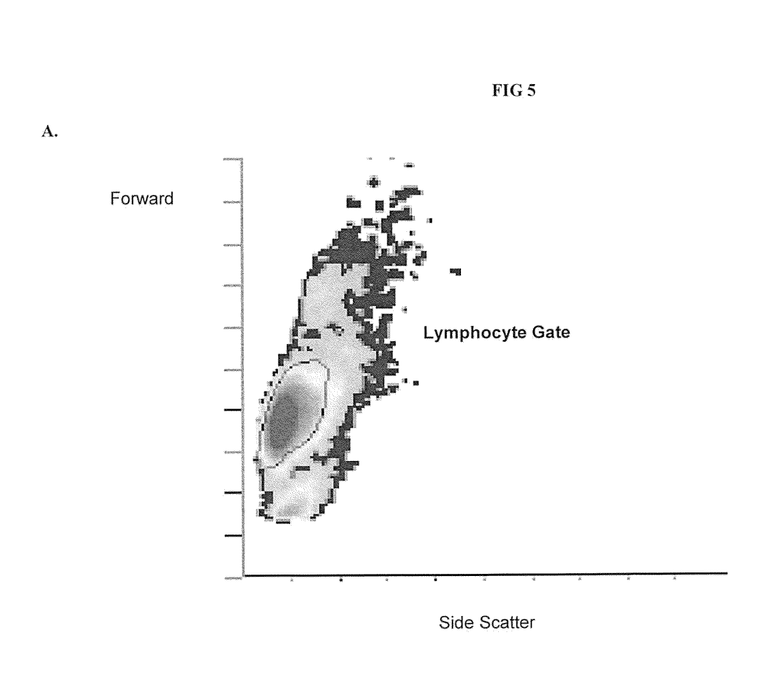

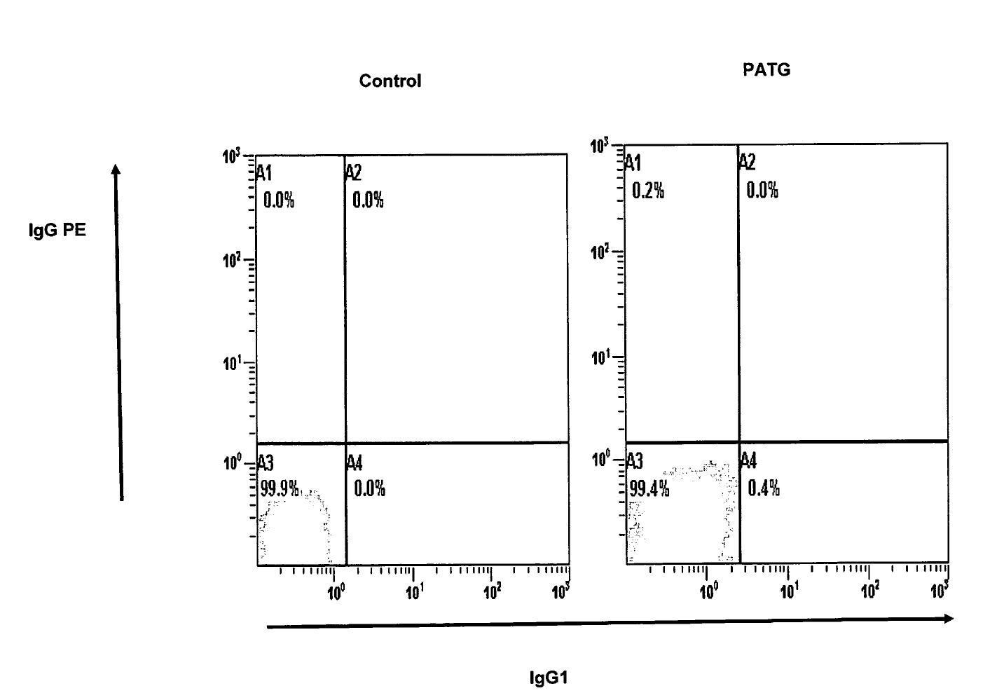

The peripheral, or circulating, and central lymphocyte response to hemorrhagic shock were examined of the experimental and control groups. Peripheral lymphocyte counts were significantly decreased in the PATG group during the hemorrhage period compared to control, p=0.001, indicating effective lymphocyte depletion (FIG. 4). During the reperfusion period, peripheral lymphocyte counts in the PATG group were not significantly different from control.

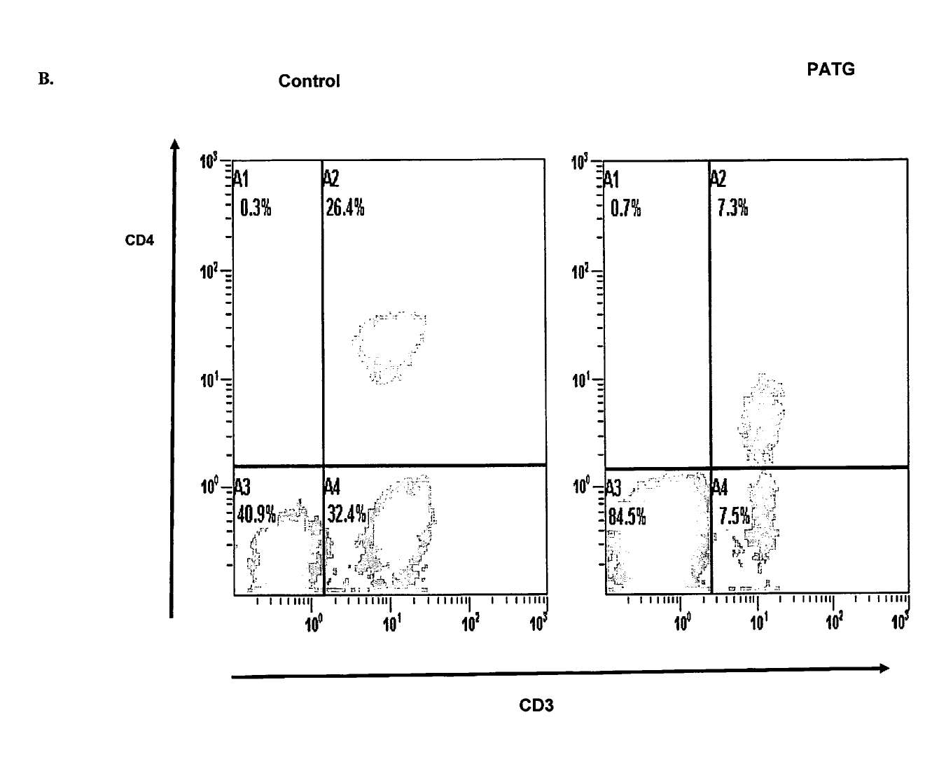

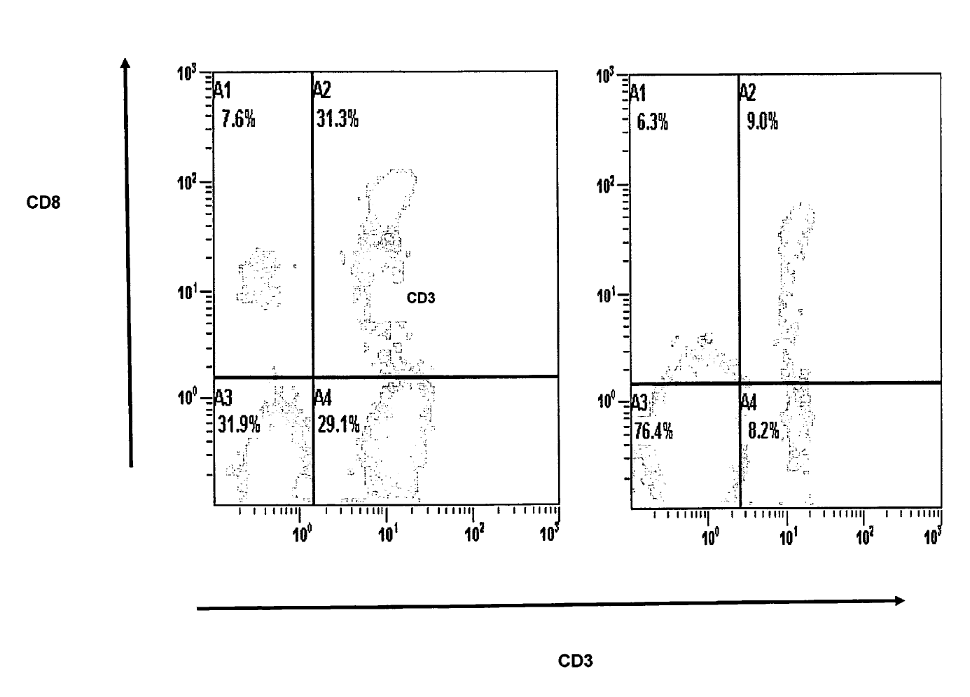

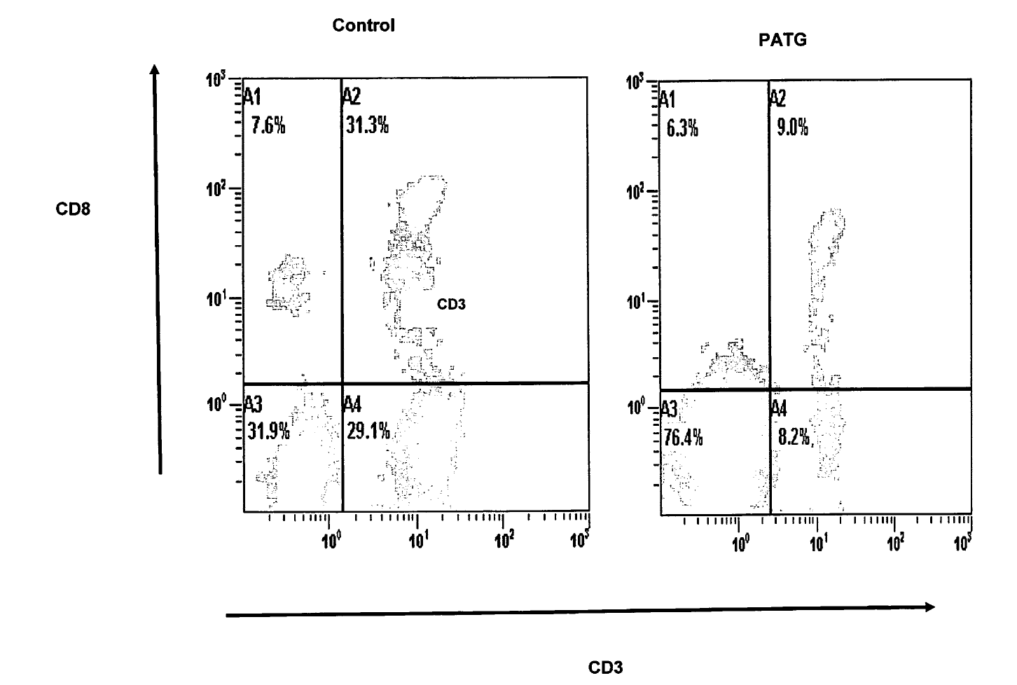

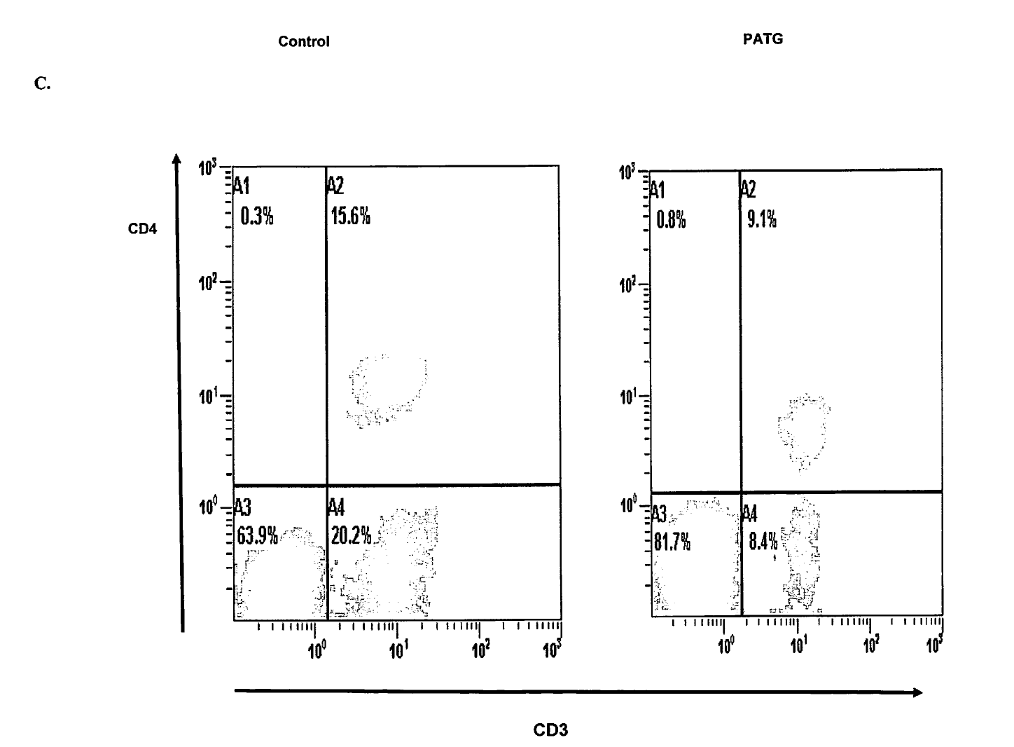

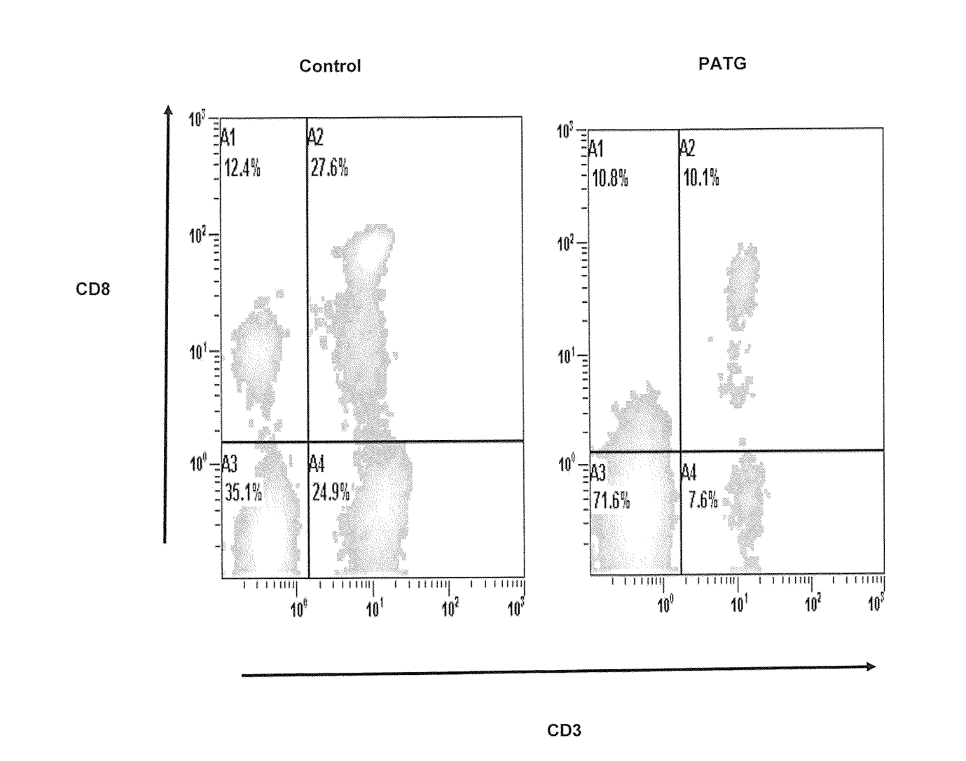

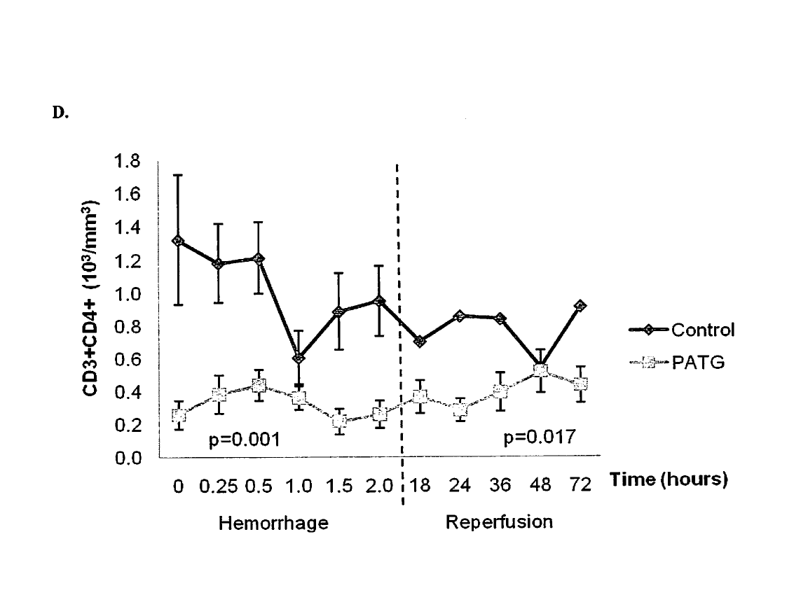

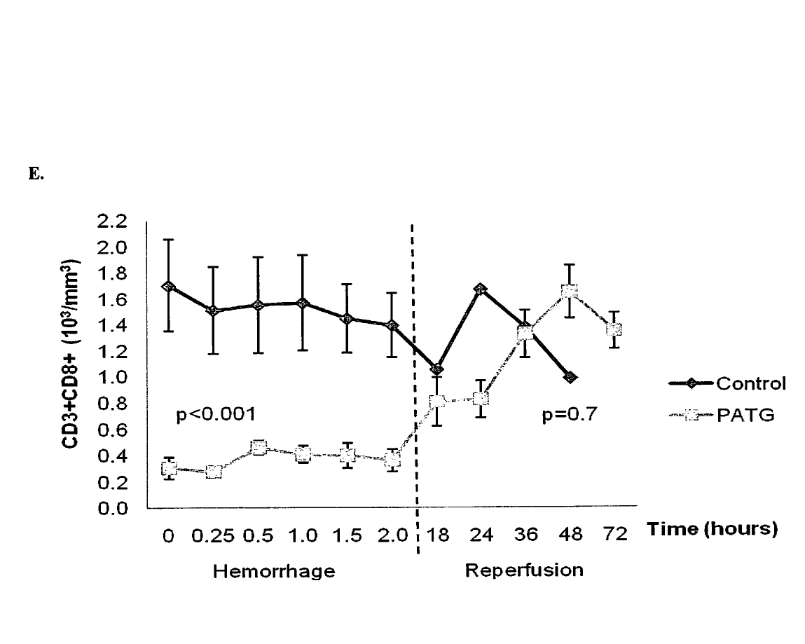

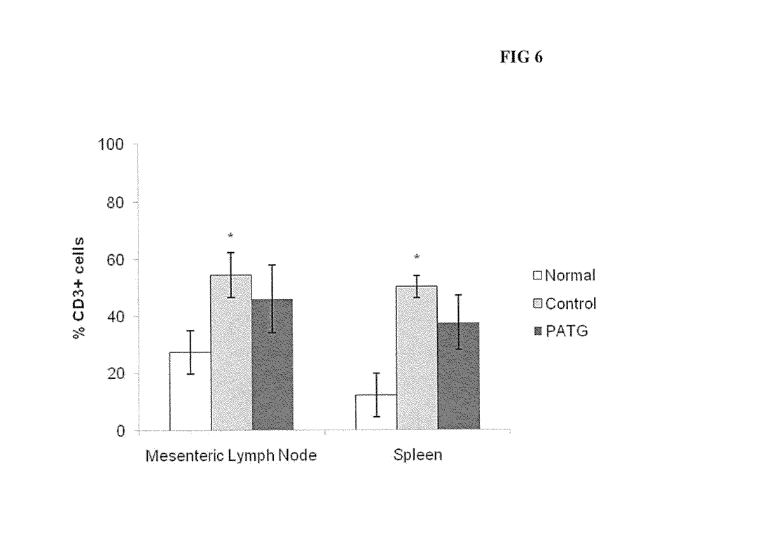

Peripheral CD4+ and CD8+ T cells were also measured in the experimental and control groups. Representative FACS plots are shown in FIG. 5. CD3+CD4+ and CD3+CD8+ lymphocytes were decreased in the PATG group at time of injury, indicating effective CD4+ and CD8+ T cell depletion (FIG. 5B). At the peak reperfusion injury period (t=24 hours), CD3+CD4+ and CD3+CD8+ lymphocytes were decreased in the PATG group, indicating sustained CD4+ and CD8+ T cell depletion (FIG. 5C).

CD3+CD4+ and CD3+CD8+ lymphocyte counts were quantified throughout hemorrhage and reperfusion periods (FIG. 5D, E). During the hemorrhage period, both CD3+CD4+ (p=0.001) and CD3+CD8+ (p<0.001) lymphocytes were significantly reduced, indicating sustained depletion. During reperfusion, CD3+CD4+ (p<0.001) lymphocytes remained significantly depleted, but CD3+CD8+ (p=0.7) lymphocytes were not different from controls.

Central lymphocytes were evaluated with CD3 immunohistochemistry staining of mesenteric lymph nodes and spleen tissue at time of necropsy. The CD3 staining was quantified, and in the PATG group, central lymphocytes were decreased compared to control but this did not reach statistical significance. Interestingly, significant central lymphocyte sequestration was evident in the control group compared to normal, unmanipulated mesenteric lymph node and spleen tissue (p<0.05). This finding suggests that some degree of lymphocyte sequestration is part of the normal response to hemorrhagic shock. PATG appeared to primarily affect peripheral lymphocytes, and in particular CD4+ T cells, under the experimental conditions.

### Peripheral and Tissue Neutrophil Response to Hemorrhagic Shock

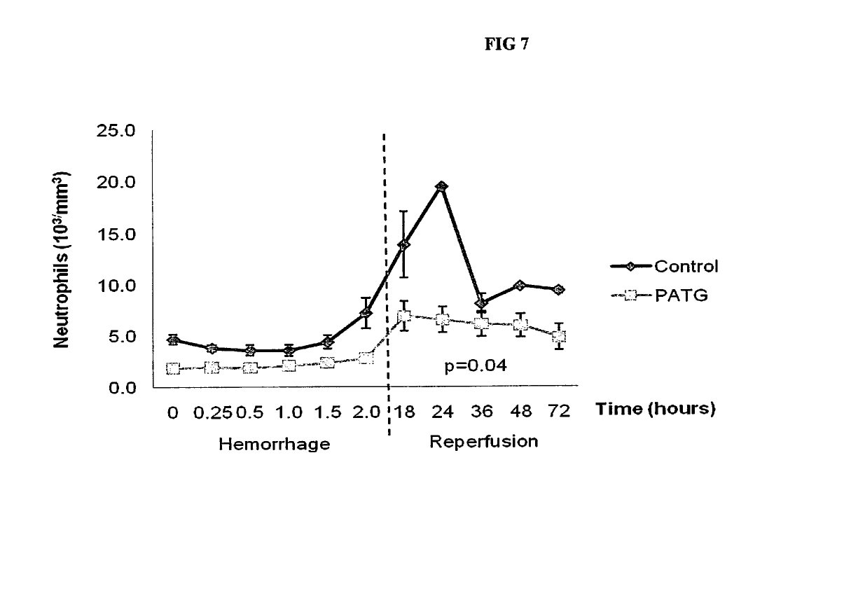

To study the innate cellular response to hemorrhagic shock, peripheral and tissue neutrophils of the experimental and control groups were examine. Throughout the hemorrhage period, there was some evidence of peripheral neutrophil depletion in the PATG group, although not statistically significant when compared to control group (FIG. 7). During reperfusion, peripheral neutrophils were markedly increased in the control group, particularly during the peak reperfusion injury period (t=24 hours). This peripheral neutrophil response was attenuated in the PATG (p=0.04) group throughout the reperfusion period.

The tissue neutrophil response to hemorrhagic shock was evaluated by examining lung tissue neutrophil infiltration. Lung is a major target organ of neutrophils following shock. There was histologic evidence of significant neutrophil infiltration in lung tissue of the control group, which was quantified with myeloperoxidase (MPO) staining. Control MPO+ cells were significantly increased compared to normal, unmanipulated lung tissues (FIG. 8). As with the peripheral neutrophils, lung tissue neutrophils were significantly decreased in the PATG (p=0.003) group compared to control. Thus, both peripheral and tissue neutrophils were attenuated in the experimental group, suggesting that lymphocyte manipulation also disrupted the innate neutrophil response to hemorrhagic shock.

### Immune Molecular Profile in Hemorrhagic Shock

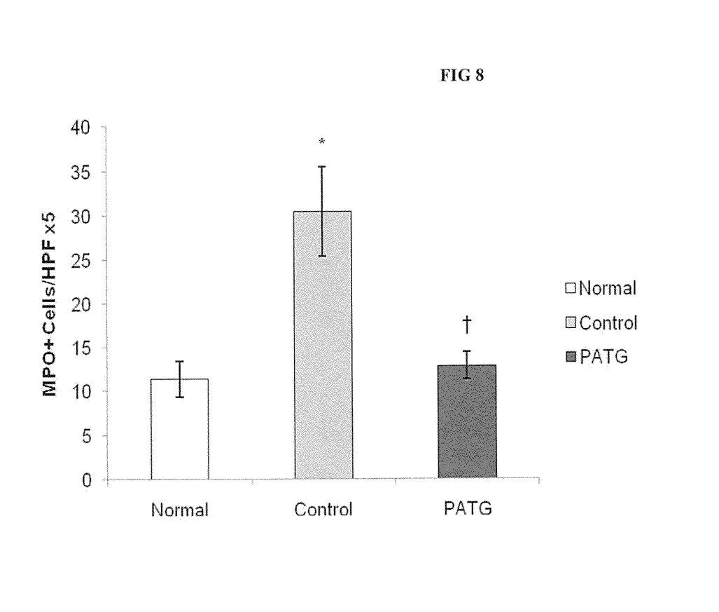

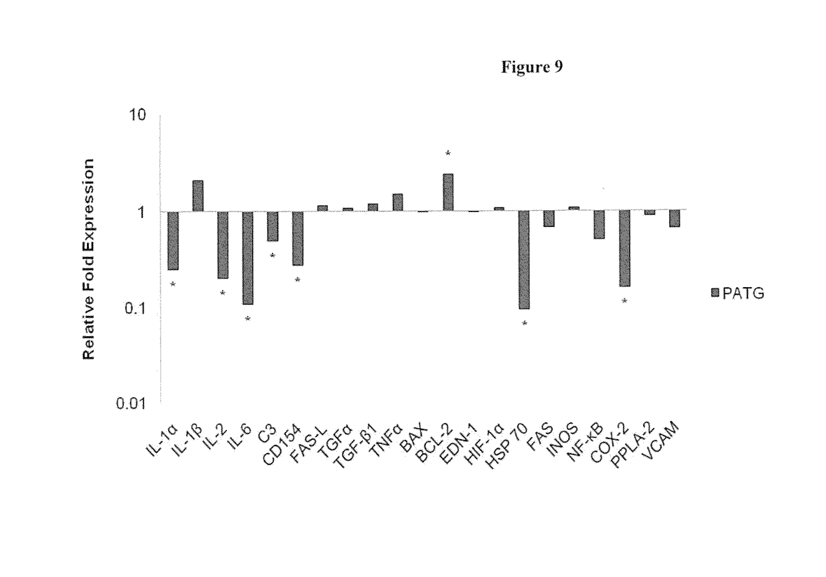

Twenty-one immune-related genes from liver tissue were analyzed, including IL-1α, IL-1β, IL-2, IL-6, C3, CD154, FAS-L, TGFα, TGF-β1, TNFα, BAX, BCL-2, EDN-1, HIF-1α, HSP70, FAS, INOS, NF-ₖB, COX-2, PPLA-2, and VCAM. Liver is a major effector organ of the systemic immune response to shock. Satisfactory mRNA was available for PATG (n=6) was compared to the control (n=5) group. Multiple immune targets, including IL-1α, IL-2, IL-6, C3, CD 154, HSP70 and COX-2, were significantly down-regulated in the PATG group relative to the control group (FIG. 9). An apoptosis gene, BCL-2, was up-regulated in the PATG group. Thus, lymphocyte depletion appeared to reduce liver inflammation following hemorrhagic shock.

### Discussion

This study shows that lymphocyte (especially T cell and B cells) depletion significantly improves reperfusion survival following experimental hemorrhagic shock in a clinically relevant large animal model. Lymphocyte depletion appears to modulate innate cellular and molecular activation following hemorrhagic shock. Specifically, disruption of the innate lymphocyte response resulted in significantly decreased circulating and lung tissue infiltrating neutrophils and decreased expression of liver immune-related genes. Lymphocytes mediate critical innate events following hemorrhage, and lymphocyte modulation may ameliorate reperfusion injury.

An important limitation in the interpretation of this study is the multitude of antibody specificities in polyclonal ATG preparations. (35) In addition to lymphocyte depletion, ATG also interferes with leukocyte-endothelium interactions by binding to adhesion molecules and chemokine receptors (36), and these effects may account for some of the observed immune protection in the experimental group. Furthermore, ATG has B lymphocyte depletional effects (37), while this study focused primarily on the contribution of only T lymphocytes. B cells have been shown to prevent IRI in multiple models and may play an important role in the postinjury immune response. (38, 39) In addition, a relatively high dose of PATG was required for >50% lymphocyte depletion. High ATG doses can result in hemolytic anemia, neutropenia, and thrombocytopenia. (15) A non-statistically significant reduction in neutrophils was observed in the experimental group, which may nonetheless confound the study results.

### EXAMPLE 2

### Lymphocyte Modulation with FTY720 Improves Hemorrhagic Shock Survival in Swine

### Animal Preparation

Male and female 3-12 month Yorkshire (Sus scrofa domestica) swine weighing 25-35 kg were acquired from ABI farms (Donsboro, Pa.). Feed was withheld 12 hours before surgery. Prior to surgery, animals were sedated and anesthesia induced with intramuscular ketamine hydrochloride (12-20 mg/kg of body weight) and xylazine (2.2 mg/kg of body weight) as well as atropine sulfate (0.05 mg/kg of body weight) to decrease tracheal secretions. Mask ventilation with Isoflurane (5.0%) was used to facilitate endotracheal intubation. Animals were ventilated (Ohmeda 7800 series ventilator, Datex, Madison, Wis.) at 12-15 breaths/min and tidal volume 10 mL/kg. Anesthesia was maintained with Isoflurane (1.5-2.5%) in 21-25% O2.

Following adequate anesthesia the right external and internal jugular veins and carotid artery were isolated. A 9Fr dual-lumen, tunneled Hickman catheter was placed in the external jugular vein for drug infusion and blood collection during the survival period. A 9Fr introducer sheath was placed in the internal jugular vein and a 7.5Fr pulmonary artery catheter (PAC; Edwards Life Sciences, Irvine, Calif.) was inserted for continuous hemodynamic monitoring. An 18G Angiocath was placed in the carotid artery and mean arterial pressure (MAP) was continuously transduced. A midline laparotomy was performed to expose the liver and isolate the left lateral lobe. Urine was collected via bladder catheterization. Rectal temperature was monitored continuously. Normothermia (37° C.) was maintained with a warming device (Model 505, Bair Hugger, Augustine Medical, Eden Prairie, Minn.).

### Liver Injury and Resuscitation:

All animal groups underwent a standardized liver injury and resuscitation protocol. (FIG. 1A.) A reproducible liver injury was created by placing a ring clamp over the left lower lobe, 50% in width and 3.5-5 mm from the apex, adjusting for the relative size of the animal. The clamp was closed and a 10 blade was used to lacerate the lobe from the top of the clamp through the remaining width. The liver injury denoted the start of the pre-hospital phase (Time 0). After 1 minute, the clamp was removed and the remaining tissue excised, resulting in a 25% lobectomy, consistent with a grade III liver injury. The hepatectomy specimen was weighed and divided by animal weight (kg) and reported as hepatectomy weight index (g/kg). Bleeding was spontaneous and removed continually via suction and measured by weight. Blood loss weight (g) was used to calculate estimated blood loss volume (%). At 15 minutes, animals were infused 250 mL normal saline (NS) over the next 60 minutes. This time point represents the arrival of a first responder (e.g. medic) who has limited carrying capacity. After 1 hour blood collection was discontinued and the abdomen packed then closed with towel clips.

After a total of 2 hours, the pre-hospital phase was completed and hospital arrival was simulated. This time point represents arrival of the patient to a hospital with surgical capabilities. The abdomen was reopened, residual blood suctioned, sponges collected, and blood loss quantified by weight. The liver injury was repaired with suture and the abdomen definitively closed. Invasive lines were removed and the carotid artery repaired. One unit of allogeneic whole blood (10 mL/kg) (Thomas Morris, Reisterstown, Md.) was administered for hemoglobin <7 g/dL, otherwise animals received additional NS at 10 mL/kg. Anesthesia was stopped and the animals extubated.

### Experimental Group:

Animals in the FTY720 group were treated 15 min after liver injury. FTY720 (0.3 mg/kg) (Cayman, Ann Arbor, Mich.) was diluted in 250 mL NS and administered over 60 min in place of the vehicle 250 mL NS infusion.

### Post-Surgical Care and Histology:

Animals were allowed to eat a regular diet and drink water following surgery. Buprenorphine (0.05-0.1 mg/kg IM/IV) was administered every 6-12 hours if the animals demonstrated any sign of pain. If the animals showed any sign of severe disability (e.g. inability to ambulate, eat, drink), severe infection, or uncontrolled pain, they were euthanized (Euthasol, Virbac AH, Fort Worth, Tex.) and taken for necropsy. At the end of the survival period (72 hours post-injury), surviving animals were euthanized and taken for necropsy. Samples of the heart, lung, liver, kidney, small intestine, mesenteric lymph node, and spleen were taken. A portion of each sample was submitted for histology. Additional tissue samples were stored in RNALater (QIAGEN®, Valencia, Calif.) and flash frozen using liquid nitrogen within 48 hours. These samples were stored at −80° C.

### Laboratory Data:

Blood samples were collected at 0, 15, 30, 60, 90, and 120 minutes during the hemorrhage period and at 18, 24, 36, 48, and 72 hours during the survival period. Complete blood counts with differentiation were obtained using a cell counter (NDvia120 Hematology System, Siemens, Deerfield, Ill.).

### Immunohistochemistry

Tissue slides were deparaffinized using xylene followed by graded baths of ethanol. A DAKO Autostainer Plus Universal Staining System (DAKO, Carpenteria, Calif.) was used for automated immunohistochemical staining. Immunohistochemical detection of CD3 was performed on sections of formalin fixed, paraffin embedded blocks of lymph node and spleen tissue. Antigen retrieval was performed using Trilogy (Cell Marque, Rocklin, Calif.) for 30 minutes. Rat, anti-human CD3, monoclonal antibody (AbD Serotec, Raleigh, N.C.) was used at a dilution of 1:50 and incubated overnight at 4 C. Biotinylated goat anti-rat IgG specific polyclonal antibody (BD Biosciences, San Jose, Calif.) was applied as a secondary antibody at a 1:100 dilution for 30 minutes at room temperature. The chromogen applied was 3,3′ Diaminobenzidine (DAKO, Carpenteria, Calif.) for 10 minutes. The sections were counterstained with Hematoxylin (DAKO, Carpenteria, Calif.). External negative controls were processed identically as CD3 but the primary antibody was substituted with normal rat serum. A tissue sample was considered positive if reactive cells, lymphocytes, demonstrated reactivity. An Automated Cellular Imaging System (ACIS) was used to quantify the CD3 immunohistochemistry staining. By using the ACIS system, CD3 immunohistochemical staining could be specifically quantitated by determining how much brown (positive CD3 immunohistochemical reaction) was in the image compared to the amount of blue (hematoxylin nuclear counterstain).

Immunohistochemical detection of MPO was performed on sections of formalin fixed, paraffin embedded blocks of lung tissue. Antigen retrieval was performed using Proteinase K (DAKO, Carpenteria Calif.) for 30 minutes. Polyclonal rabbit, anti-human MPO (DAKO, Carpenteria Calif.) was used at a ready to use dilution and incubated at room temperature for 30 minutes. Envision, anti-rabbit (DAKO, Carpenteria Calif.) was applied as a secondary antibody for 30 minutes at room temperature. The chromogen applied was 3,3′ Diaminobenzidine (DAKO, Carpenteria, Calif.) for 10 minutes. The sections were counterstained with Hematoxylin (DAKO, Carpenteria, Calif.). External negative control was processed identically as with MPO but the primary antibody was substituted with normal rabbit serum. A tissue sample was considered positive if reactive cells, neutrophils, demonstrated reactivity in conjunction with a segmented nucleus. Neutrophils were noted mainly in circulation in small septal vessels and in large vessels. Cells with appropriate nuclear morphology and the presence of light to intensely reactive granules were counted; 5 random 40× fields were assessed.

### RNA Extraction and Quantitative Real-Time Polymerase Chain Reaction

Total RNA was isolated from liver tissue taken at necropsy using Qiagen RNeasy Mini Kit (QIAGEN® Inc. Valencia, Calif.) according to manufacturer's instructions. RNA purity and quantity were assessed by measuring the A₂₆₀, A₂₈₀, and A₂₃₀ on a Nanodrop Spectrophotometer (NanoDrop Technologies Inc. Wilmington, Del.). RNA quality was determined from the 28S/18S rRNA ratio and RNA Integrity Number (RIN) using an Agilent 2100 BioAnalyzer (Agilent Technologies Inc. Santa Clara, Calif.). RIN values for all specimens in this study were ≧6.5. Satisfactory mRNA was available for FTY720 (n=7) and the control (n=5) groups. Reverse transcriptions were performed using Roche 1^{st} Strand Synthesis kits (Roche Diagnostics Corporation, Indianapolis, Ind.) according to the manufacturer's protocol. Quantitative real-time polymerase chain reaction (QRT-PCR) was performed using the 7900HT Fast Real-Time PCR System (Applied Biosystems, Foster City, Calif.) to assess mRNA transcript expression of 21 immune-related genes. Taqman chemistry was used. Primers for 18S rRNA target were used as an internal control for each reaction. Primers and probes for the targets of interest were obtained from Applied Biosystems (Table 1). All samples were run in duplicate. Individual samples were compared to averaged control tissue expression. Transcript quantification was derived using the comparative threshold cycle method and reported as the mean n-fold difference of the experimental sample to the control pool.

### Statistical Analysis:

Animals were randomly assigned to the control or experimental groups. Survival analysis between groups was performed by Kaplan Meier survival plots with the log rank test. Continuous data was compared with the Student t-test or nonparametric tests as appropriate. Multiple comparisons were performed with one-way ANOVA. A one-way ANOVA with repeated measures design was used for continuous time-dependent comparisons. Statistical analysis was performed using SPSS (SPSS Inc., Chicago, Ill.). A two-tailed p value <0.05 was considered statistically significant. All data is represented as means ±standard error (SEM) unless otherwise specified.

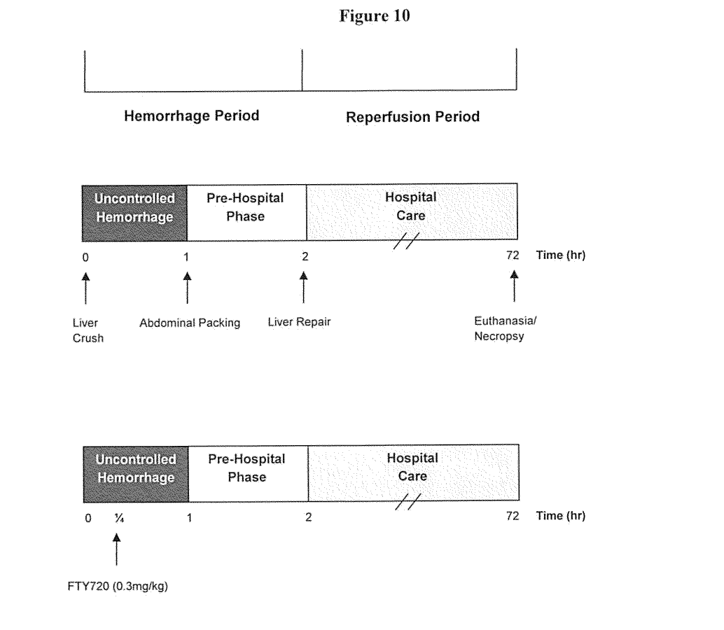

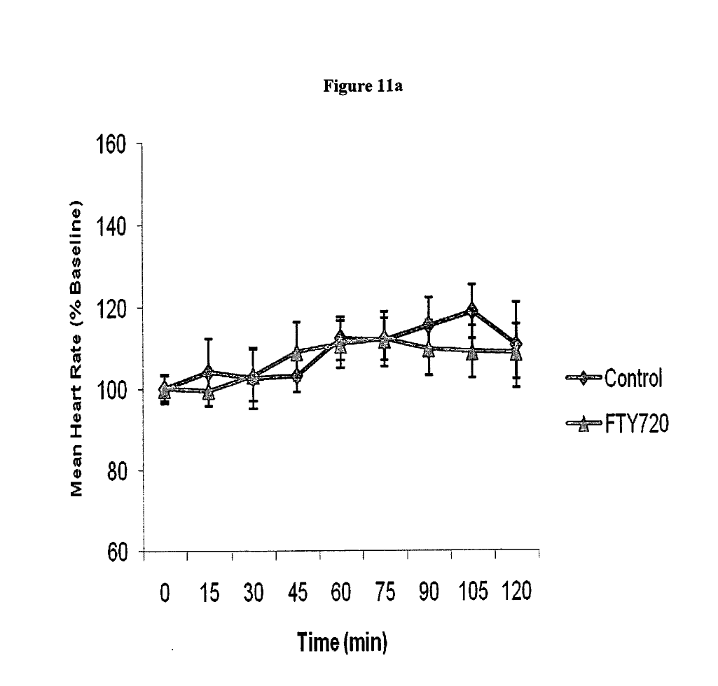

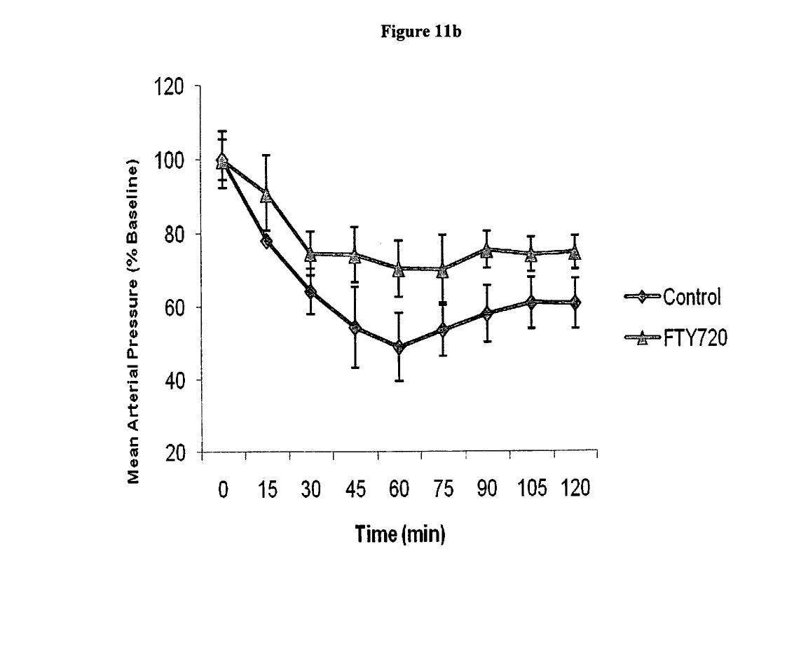

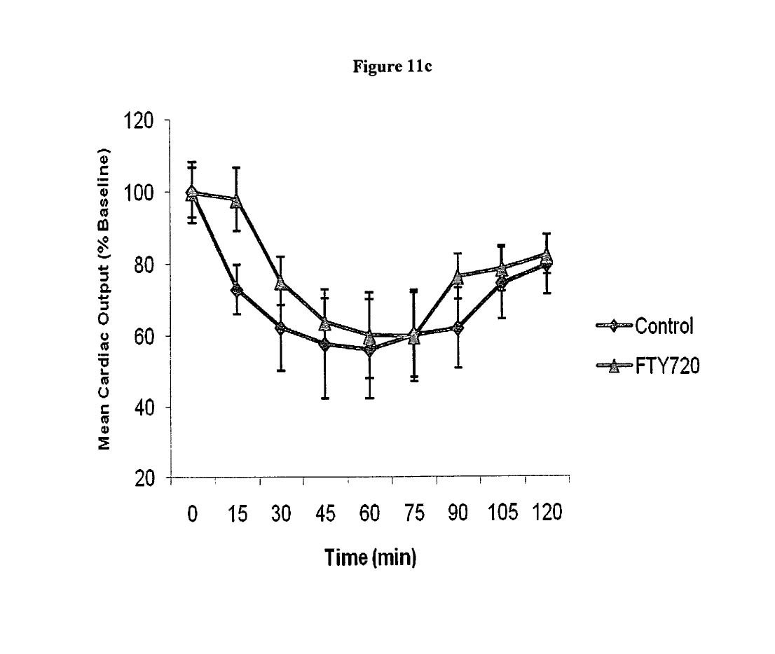

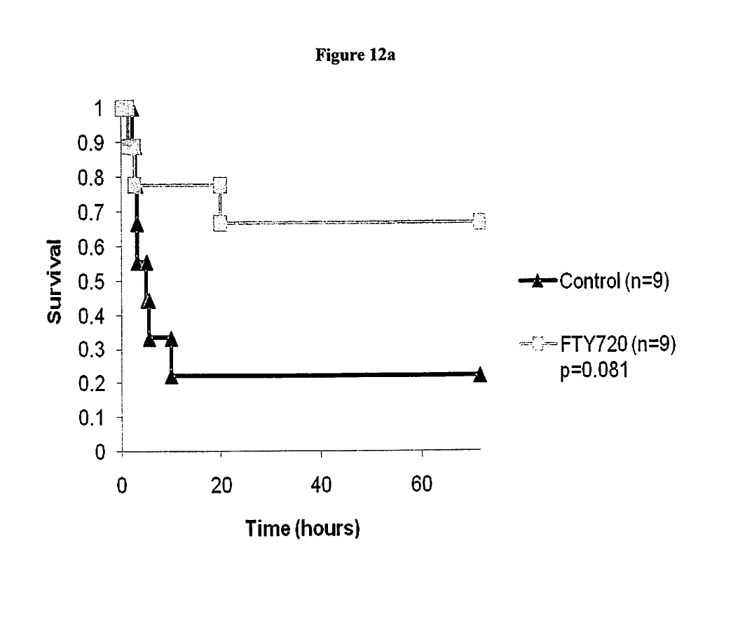

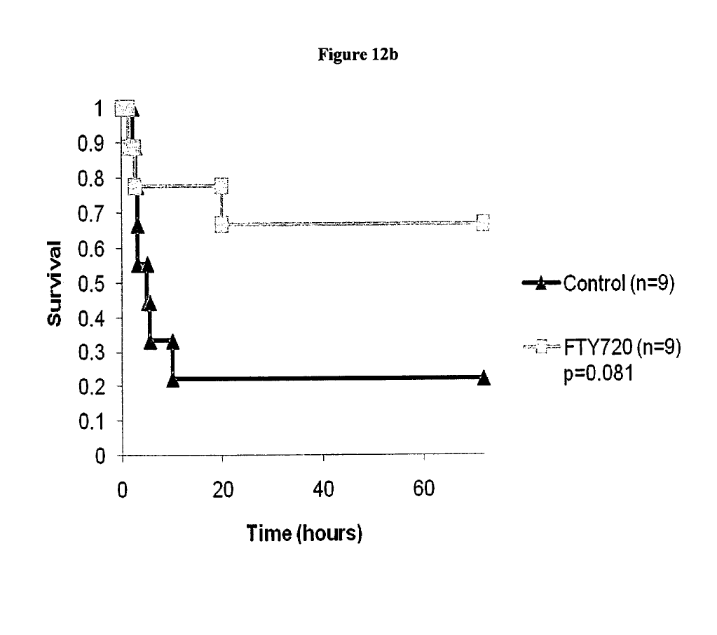

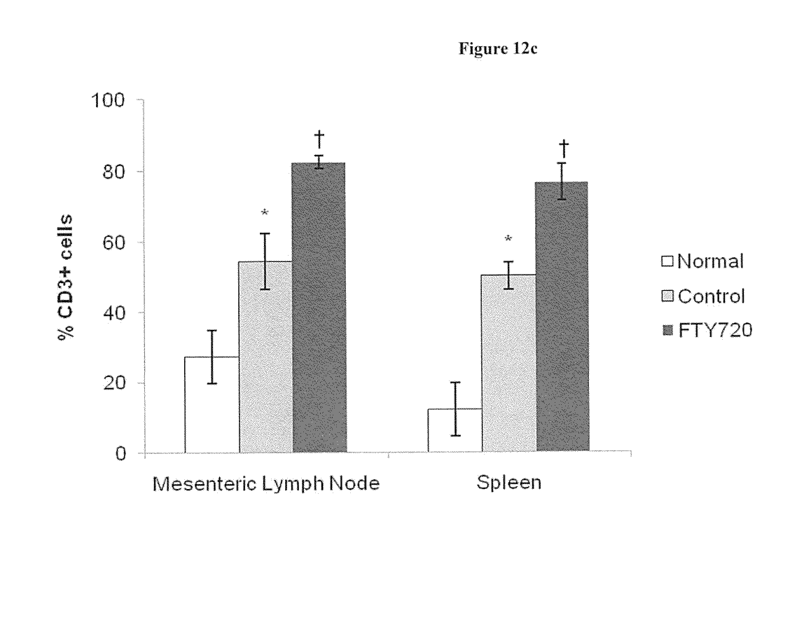

Despite similar liver injury delivery, percent mean blood loss volume was lower in the FTY720 group compared to control but this was not statistically significant. Importantly, invasive hemodynamic monitoring during the hemorrhage period demonstrated shock physiology in all animal groups (FIG. 10). Overall survival in the experimental groups was improved compared to control although not statistically significant (FIG. 12a). During the reperfusion period (following hemorrhage) survival was statistically improved in the FTY720 group compared to control (FIG. 12b). Thereby, disruption of the lymphocyte response to shock during hemorrhage appeared to significantly reduce mortality associated with reperfusion injury.

CD3 immunohistochemistry staining of mesenteric lymph nodes and spleen tissue at time of necropsy to evaluate central lymphocyte sequestration were performed. There was significant central lymphocyte sequestration in the FTY720 group compared to control in both mesenteric lymph node and spleen (FIG. 12c). Interestingly, significant central lymphocyte sequestration was evident in the control group compared to naïve swine mesenteric lymph node and spleen tissue. This finding suggests that some degree of lymphocyte sequestration is part of the normal response to hemorrhagic shock. Importantly, FTY720 mediated lymphocyte sequestration and conferred a survival advantage in this liver injury model.

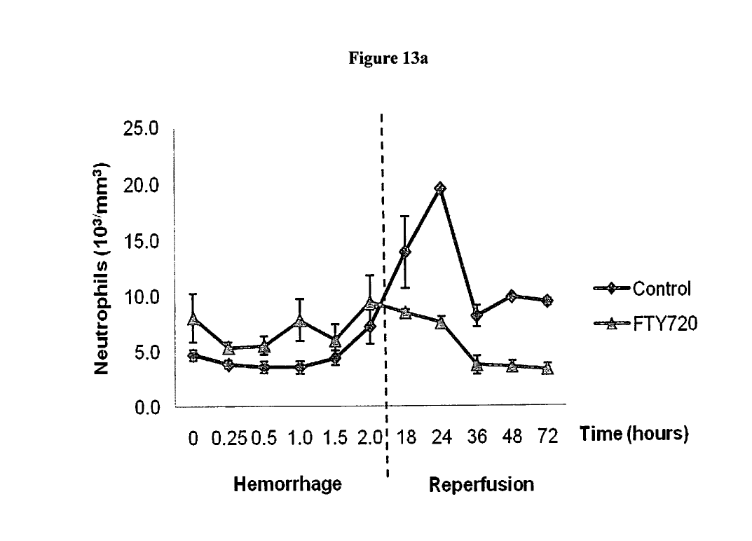

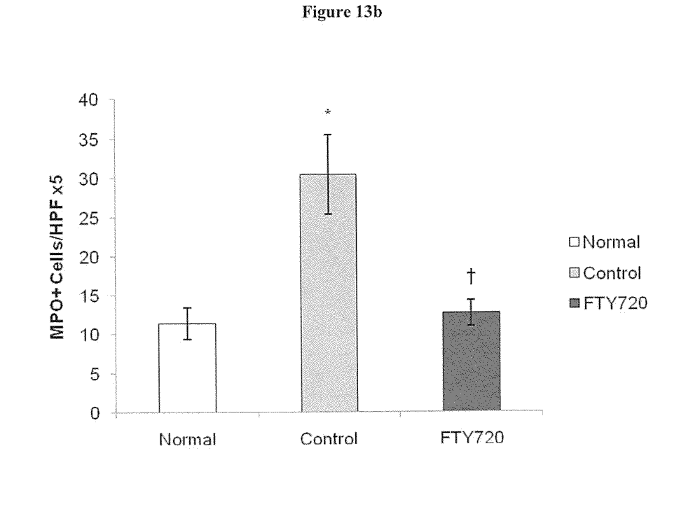

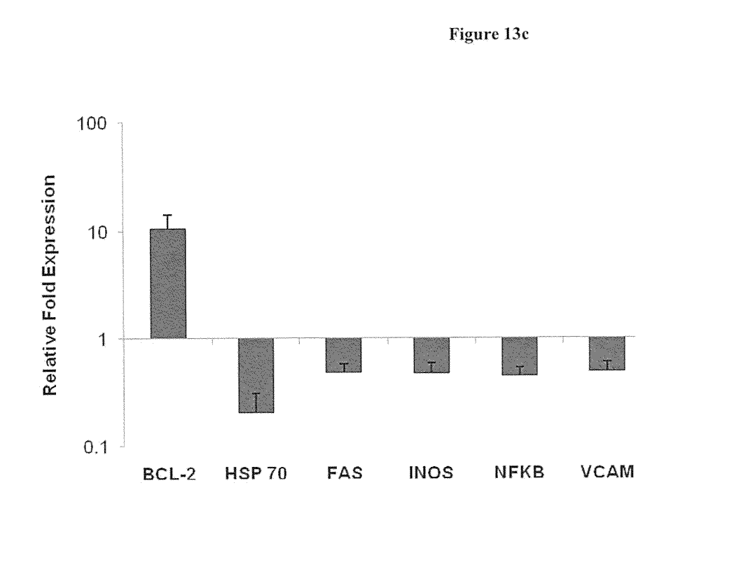

In order to study the innate cellular response to hemorrhagic shock, peripheral blood and tissue neutrophil levels in the FTY720 and control groups were examine. Neutrophils have been established as mediators of injury following hemorrhage in both animal and human studies. ¹⁰ During reperfusion, peripheral blood neutrophil numbers were markedly increased in the control group (FIG. 13a). This peripheral neutrophil response was attenuated in the FTY720 group throughout the reperfusion period. The tissue neutrophil response to hemorrhagic shock was evaluated by examining lung tissue neutrophil infiltration, a major target organ of neutrophils following shock. ¹¹ Neutrophil infiltration (H&E stained sections and immunohistochemistry using anti-MPO antibody) in lung tissue was significantly increased in control animals when compared to naïve lung tissue (FIG. 13b). In contrast, lung tissue neutrophils were significantly decreased in the FTY720 group compared to control. These findings indicate that both the peripheral blood and tissue neutrophil response was attenuated in the FTY720 group, suggesting that lymphocyte manipulation also disrupted the innate neutrophil response to hemorrhagic shock.

In order to evaluate the innate molecular response to hemorrhagic shock, gene transcripts of 21 inflammatory targets associated with IRI from liver tissue, were examined. Liver is a major effector organ of the systemic immune response to shock (Table 2). ¹² Expression of six gene targets were statistically different from the control group when compared to the FTY720 group; the anti-apoptotic gene BCL-2 was up-regulated and the apoptotic gene FAS was down regulated; innate immunity genes HSP70, INOS, and NFκB were down-regulated; the adhesion gene VCAM was also down-regulated compared to control animals (FIG. 13c). Thus, lymphocyte modulation also appeared to reduce apoptotsis and liver inflammation at the molecular level following hemorrhagic shock.

Lymphocyte sequestration with FTY720 significantly improves reperfusion survival following experimental hemorrhagic shock in a large animal model. Lymphocyte immunomodulation appears to attenuate innate cellular and molecular activation following hemorrhagic shock. Specifically, disruption of the innate lymphocyte response resulted in significantly decreased circulating and lung tissue infiltrating neutrophils and decreased expression of liver inflammatory genes. In conclusion, lymphocyte sequestration with FTY720 offers a novel and clinically applicable approach towards abrogating immune mediated reperfusion injury in trauma and surgical patients.

### PROPHETIC EXAMPLE 3

### Observation Study of Suppression of Lymphocytes for Recurrent Bowel Adhesions

### Patient Selection and Preparation

Clinically stable, non-septic patients presenting with suspected intraabdominal adhesive disease will be considered for enrollment into the clinical trial. Each potential enrollee will undergo general evaluation, hospital admission, fluid resuscitation and the verification of a bowel obstruction requiring operative intervention. Baseline renal function will be assessed determining the estimated creatinine clearance using the MDRD equation (67). Viral serologic testing to include CMV, EBV, HIV, hepatitis B and C virus will be performed. Patients having a creatinine clearance greater than 60 ml/min with appropriate viral status will serve as the study population.

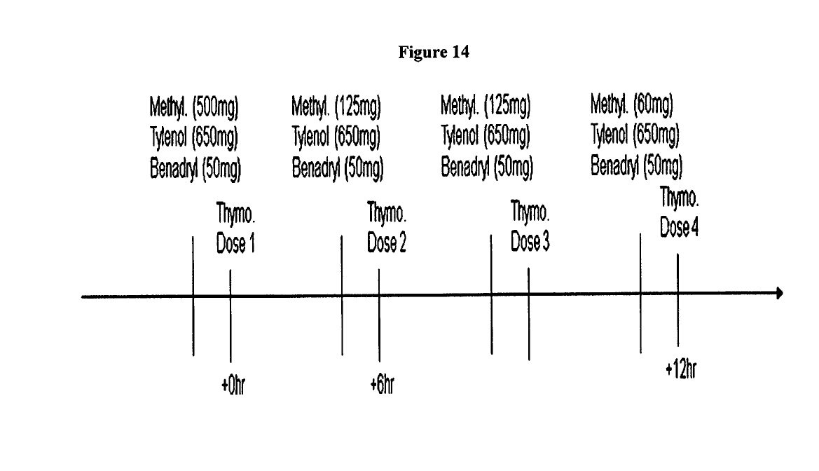

Up to 20 patients with acceptable renal function will be included in the treatment group the study. Patients will be enrolled in cohorts of 5 to assess for safety. Central venous access will be obtained for Thymoglobulin infusion. At laparotomy, and upon operative confirmation of matted adhesive disease, Thymoglobulin will be administered at 1.25 mg/kg of body weight in 4 doses (see schema FIG. 14). Laparotomy will follow standard surgical guidelines and may include bowel resection or ostomy placement if indicated. Doses 2-4 will be 1.25 mg/kg of body weight, and be given on postoperative days 1-3 respectively according to the standard prescribing guidelines for organ transplantation. All doses will be administered over 6-12 hours. Methylprednisolone 500 mg, Tylenol 650 mg, and Benadryl 50 mg will be given as a pre-medication dose before the first dose of Thymoglobulin. Methylprednisolone 125 mg, Tylenol 650 mg, and Benadryl 50 mg will be given as premedication before doses 2 and 3. Dose 4 will be preceded by methylprednisolone 60, Tylenol 650 mg and Benadryl 50 mg. In the event that dose reduction or elimination of Thymoglobulin is required, make-up doses to achieve the targeted 5 mg/kg total dose (for comparison standard solid organ transplantation induction dose is 10 mg/kg over divided doses) will be encouraged at the discretion of the attending physician.

Oral tacrolimus therapy will be initiated on post operative day 1 at a dose of 4 mg every 12 hours to be adjusted to maintain a trough blood level of 4-6 ng/ml (for comparison standard solid organ transplantation early trough levels are 8 to 12 ng/ml). Tacrolimus levels will be drawn daily while the patient is an inpatient, and as part of the patient's periodic laboratory evaluation in keeping with the participating centers' standard of care. Therapy with tacrolimus will be discontinued at 6 months or if the baseline creatinine clearance is reduced by 30% at any time point as compared to the enrollment creatinine clearance. Toxicity attributable to tacrolimus such as renal insufficiency, tremor or insulin resistance will prompt dose reduction in 25% aliquots to address clinical concerns in this regard. If drug side effects cannot be controlled trough dose reduction, patient will be considered intolerant to the drug and it will be discontinued.

Patients will receive antiviral prophylaxis in keeping with guidelines for the use of Thymoglobulin in the transplant setting, typically a 3 month course of Acyclovir (CMV seronegative patients) or Ganciclovir (CMV seropositive patients) will be transitioned to oral valacyclovir or valganciclovir, respectively. PCP prophylaxis will be employed, typically Bactrim, 1 double strength tablet given 3 times weekly. Perioperative antibacterial antibiotic prophylaxis will follow the standard of care for surgical adhesiolysis.

### Postoperative Care and Monitoring

Patients will be cared for in keeping with the standard of care for patients recovering from surgical exploration with respect to wound care, antibiotic prophylaxis, nutrition, ambulation, isolation, discharge, convalescence and rehabilitation. Physiological monitoring will follow the standard of care for patients recovering from surgical exploration but also include daily serum creatinine determination, urinalysis, CBC with differential, and tacrolimus level 12-hour trough monitoring for the first 4 postoperative days, weekly for 4 weeks, monthly for 6 months and at months 9 and 12. CMV and EBV antigenemia will be assessed by PCR at day 7, week 4, monthly for 6 months, and at months 9 and 12. Evidence of viral reactivation either clinically or based on PCR testing will prompt Tacrolimus dose reduction and/or elimination in keeping with the standard of care for transplant patients. Failure to respond to dose elimination will prompt therapeutic intervention to include anti-viral medication in keeping with the standard of care. Lymphocyte repopulation will be assessed by flow cytometry at 1 month, 6 months and at 1 year by analysis of 7 ml of heparinized blood.

Patients enrolled in the control group will be followed for observational purposes only. Patients will be contacted at similar time points as listed above (monthly from months 1 through 6 and then at months 9, 12 and 36) to assess intervening illness and recurrence of bowel obstruction. Specific complications queried via phone interview will comprise those of the composite endpoint of major wound complications (infection requiring hospitalization, fistulization or dehiscence), opportunistic infection or latent herpesviral reactivation (CMV, EBV, VZV) requiring hospitalization, malignancy, or death. In addition, the incidence of rehospitalization for recurrent adhesive disease related complications (bowel obstruction, or related complications) will be collected.

### Assessment of Adhesive Disease

Patients will be followed postoperatively in accordance with the standard of care with regard to all aspects of post-operative assessment for adhesive disease. Patients requiring readmission will be assessed by the general surgical team for assessment of operative concerns, and by the transplant surgeon on call with regard to immunosuppressive management. All patients requiring reoperation for adhesive disease will be withdrawn from tacrolimus therapy.

Those patients avoiding reoperation for 1 year from their initial operation will be offered operative assessment of adhesive disease with laparoscopy. Abdominal access will be obtained as in laparoscopic ventral hernia repair using a left subcostal open approach to place a single 5 mm trocar. Adhesions will be scored. This approach allows for safe access to the reoperative abdomen and uses a 5 mm trocar with minimal risk of subsequent hernia formation. Adhesions occurring at the site of Trocar placement are localized to the trocar site high in the abdomen and as such are not typically associated with intestinal complications. A similar model consisting of placement of a laparoscope at the time of ostomy reversal has been previously validated for the assessment of adhesion prevention using barrier agents. The video from this procedure will be recorded for independent, blinded grading of the adhesions by two separate reviewers.

The following scoring system will be used to grade adhesions by assessing incidence, type and severity of adhesions and a combined adhesive score (CAS) will be calculated for each subject (68).

TABLE 3
Incidence & Severity

| Level | Definition |
|---|---|
| 0 | No adhesions |
| 1 | Mild: covering 25% of the abdomen |
| 2 | Moderate: covering 25-60% of the abdomen |
| 3 | Severe: covering 61-75% of the abdomen |
| 4 | Extreme: covering more than 75% of the abdomen |

TABLE 4
Type of adhesion

| Level | Definition |
|---|---|
| 0 | None |
| 1 | Filmy, avascular |
| 2 | Vascular and/or dense |
| 3 | Cohesive or matted adhesions |

### REFERENCES

1. Moore F A, Moore E E. Postinjury multiple organ failure. Trauma, 5th ed. New York: McGraw-Hill; 2004.

2. Harlan J M, Winn R K. Leukocyte-endothelial interactions: clinical trials of anti-adhesion therapy. Crit Care Med 2002;30(5 Suppl):S214-219.

3. Rhee P, Morris J, Durham R, et al. Recombinant humanized monoclonal antibody against CD18 (rhuMAb CD18) in traumatic hemorrhagic shock: results of a phase II clinical trial. Traumatic Shock Group. J Trauma 2000;49(4):611-619; discussion 619-620.

4. Yonekawa K, Harlan J M. Targeting leukocyte integrins in human diseases. J Leukoc Biol 2005;77(2):129-140.

5. De A K, Kodys K M, Pellegrini J, et al. Induction of global anergy rather than inhibitory Th2 lymphokines mediates posttrauma T cell immunodepression. Clin Immunol 2000;96(1):52-66.

6. Spolarics Z, Siddiqi M, Siegel J H, et al. Depressed interleukin-12-producing activity by monocytes correlates with adverse clinical course and a shift toward Th2-type lymphocyte pattern in severely injured male trauma patients. Crit Care Med 2003;31(6):1722-1729.

7. Bandyopadhyay G, De A, Laudanski K, et al. Negative signaling contributes to T-cell anergy in trauma patients. Crit Care Med 2007;35(3):794-801.

8. Rabb H, Daniels F, O'Donnell M, et al. Pathophysiological role of T lymphocytes in renal ischemia-reperfusion injury in mice. Am J Physiol Renal Physiol 2000;279(3):F525-531.

9. Horie Y, Wolf R, Chervenak R P, et al. T-lymphocytes contribute to hepatic leukostasis and hypoxic stress induced by gut ischemia-reperfusion. Microcirculation 1999;6(4):267-280.

10. Zwacka R M, Zhang Y, Halldorson J, et al. CD4(+) T-lymphocytes mediate ischemia/reperfusion-induced inflammatory responses in mouse liver. J Clin Invest 1997;100(2):279-289.

11. Le Moine O, Louis H, Demols A, et al. Cold liver ischemia-reperfusion injury critically depends on liver T cells and is improved by donor pretreatment with interleukin 10 in mice. Hepatology 2000;31(6):1266-1274.

12. Monaco A P, Abbott W M, Othersen H B, et al. Antiserum to lymphocytes: prolonged survival of canine renal allografts. Science 1966;153(741):1264-1267.

13. Starzl T E. Heterologous antilymphocyte globulin. N Engl J Med 1968;279(13):700-703.

14. Metchnikoff E. Etude sur la resorption des cellules. Ann inst Pasteur 1899;13:737.

15. Preville X, Flacher M, LeMauff B, et al. Mechanisms involved in antithymocyte globulin immunosuppressive activity in a nonhuman primate model. Transplantation 2001;71(3):460-468.

16. Raefsky E L, Gascon P, Gratwohl A, et al. Biological and immunological characterization of ATG and ALG. Blood 1986;68(3):712-719.

17. Genestier L, Fournel S, Flacher M, et al. Induction of Fas (Apo-1, CD95)-mediated apoptosis of activated lymphocytes by polyclonal antithymocyte globulins. Blood 1998;91(7):2360-2368.

18. Bonnefoy-Berard N, Vincent C, Revillard J P. Antibodies against functional leukocyte surface molecules in polyclonal antilymphocyte and antithymocyte globulins. Transplantation 1991;51(3):669-673.

19. Arnaud F, Handrigan M, Hammett M, et al. Coagulation patterns following haemoglobin-based oxygen carrier resuscitation in severe uncontrolled haemorrhagic shock in swine. Transfus Med 2006;16(4):290-302.

20. Livak K J, Schmittgen T D. Analysis of relative gene expression data using real-time quantitative PCR and the 2(-Delta Delta C(T)) Method. Methods 2001;25(4):402-408.

21. Lenz A, Franklin G A, Cheadle W G. Systemic inflammation after trauma. Injury 2007;38(12):1336-1345.

22. Matzinger P. An innate sense of danger. Semin Immunol 1998;10(5):399-415.

23. Kokura S, Wolf R E, Yoshikawa T, et al. Postanoxic T lymphocyte-endothelial cell interactions induce tumor necrosis factor-alpha production and neutrophil adhesion: role of very late antigen-4/vascular cell adhesion molecule-1. Circ Res 2000;86(12):1237-1244.

24. Kokura S, Wolf R E, Yoshikawa T, et al. Endothelial cells exposed to anoxia/reoxygenation are hyperadhesive to T-lymphocytes: kinetics and molecular mechanisms. Microcirculation 2000;7(1):13-23.

25. Burne M J, Daniels F, El Ghandour A, et al. Identification of the CD4(+) T cell as a major pathogenic factor in ischemic acute renal failure. J Clin Invest 2001;108(9):1283-1290.

26. Takada M, Chandraker A, Nadeau K C, et al. The role of the B7 costimulatory pathway in experimental cold ischemia/reperfusion injury. J Clin Invest 1997;100(5):1199-1203.

27. Beiras-Fernandez A, Thein E, Chappel D, et al. Polyclonal anti-thymocyte globulins influence apoptosis in reperfused tissues after ischaemia in a non-human primate model. Transpl Int 2004;17(8):453-457.

28. Mehrabi A, Mood Z h A, Sadeghi M, et al. Thymoglobulin and ischemia reperfusion injury in kidney and liver transplantation. Nephrol Dial Transplant 2007;22 Suppl 8:viii54-viii60.

29. Revillard J P. The search for disease susceptibility genes. Medical and ethical problems of predictive medicine. C R Acad Sci III 1999;322(10):825-829.

30. Rabb H, O'Meara Y M, Maderna P, et al. Leukocytes, cell adhesion molecules and ischemic acute renal failure. Kidney Int 1997;51(5):1463-1468.

31. Botha A J, Moore F A, Moore E E, et al. Early neutrophil sequestration after injury: a pathogenic mechanism for multiple organ failure. J Trauma 1995;39(3):411-417.

32. Weiss S J. Tissue destruction by neutrophils. N Engl J Med 1989;320(6):365-376.

33. Pallister I, Dent C, Topley N. Increased neutrophil migratory activity after major trauma: a factor in the etiology of acute respiratory distress syndrome? Crit Care Med 2002;30(8):1717-1721.

34. Windsor A C, Mullen P G, Fowler A A, et al. Role of the neutrophil in adult respiratory distress syndrome. Br J Surg 1993;80(1):10-17.

35. Rebellato L M, Gross U, Verbanac K M, et al. A comprehensive definition of the major antibody specificities in polyclonal rabbit antithymocyte globulin. Transplantation 1994;57(5):685-694.

36. Michallet M C, Preville X, Flacher M, et al. Functional antibodies to leukocyte adhesion molecules in antithymocyte globulins. Transplantation 2003;75(5):657-662.

37. Bonnefoy-Berard N, Genestier L, Flacher M, et al. Apoptosis induced by polyclonal antilymphocyte globulins in human B-cell lines. Blood 1994;83(4):1051-1059.

38. Burne-Taney M J, Ascon D B, Daniels F, et al. B cell deficiency confers protection from renal ischemia reperfusion injury. J Immunol 2003;171(6):3210-3215.

39. Burne-Taney M J, Yokota-Ikeda N, Rabb H. Effects of combined T- and B-cell deficiency on murine ischemia reperfusion injury. Am J Transplant 2005;5(6):1186-1193.

40. Weaver T A, Kirk A D. Alemtuzumab. Transplantation 2007;84(12):1545-1547.

41. Dragun D, Bohler T, Nieminen-Kelha M, et al. FTY720-induced lymphocyte homing modulates post-transplant preservation/reperfusion injury. Kidney Int 2004;65(3):1076-1083.

42. Delbridge M S, Shrestha B M, Raftery A T, et al. Reduction of ischemia-reperfusion injury in the rat kidney by FTY720, a synthetic derivative of sphingosine. Transplantation 2007;84(2):187-195.

43. Barkhausen T, Frerker C, Pütz C, Pape H C, Krettek C, van Griensven M. Depletion of NK cells in a murine polytrauma model is associated with improved outcome and a modulation of the inflammatory response. Shock. 2008 October;30(4):401-10.

44. Lenz, A., Franklin, G. A., and Cheadle, W. G., Injury 38 (12), 1336 (2007).

45. Moore, F. A. and Moore, E. E., Postinjury multiple organ failure., Trauma, 5th ed. (McGraw-Hill, New York, 2004).

46. Huang, Y., Rabb, H., and Womer, K. L., Cell Immunol 248 (1), 4 (2007).

47. Zwacka, R. M. et al., J Clin Invest 100 (2), 279 (1997).

48. Burne, M. J. et al., J Clin Invest 108 (9), 1283 (2001); Le Moine, O. et al., Hepatology 31 (6), 1266 (2000); Takada, M. et al., J Clin Invest 100 (5), 1199 (1997).

49. Rivera, J., Proia, R. L., and Olivera, A., Nat Rev Immunol 8 (10), 753 (2008).

50. Brinkmann, V., Cyster, J. G., and Hla, T., Am J Transplant 4 (7), 1019 (2004).

51. Dragun, D. et al., Kidney Int 65 (3), 1076 (2004).

52. Arnaud, F. et al., Transfus Med 16 (4), 290 (2006); Hawksworth, J. S. et al., J Thromb Haemost 7 (10), 1663 (2009).

53. Rabb, H. et al., Kidney Int 51 (5), 1463 (1997); Botha, A. J. et al., J Trauma 39 (3), 411 (1995); Weiss, S. J., N Engl J Med 320 (6), 365 (1989).

54. Pallister, I., Dent, C., and Topley, N., Crit Care Med 30 (8), 1717 (2002); Windsor, A. C., Mullen, P. G., Fowler, A. A., and Sugerman, H. J., Br J Surg 80 (1), 10 (1993).

55. Horie, Y. et al., Microcirculation 6 (4), 267 (1999).

56. Vrijland W W, Jeekel J, van Geldrop H J et al. Abdominal adhesions: intestinal obstruction, pain, and infertility. Surgical Endoscopy 2003;17:1017-22.

57. Ellis H. The causes and prevention of intestinal adhesions. British Journal of Surgery. 1982;69:241-243.

58. Miller G, Boman J, Shrier I et al. Natural history of patients with adhesive small obstruction. British Journal of Surgery. 2000;87:1240-1247.

59. Menzies D, Ellis H. Instestinal obstruction from adhesions—how big is the problem? Annals Royal College Surgeons England 1990;72:60-3.

60. Wilson M S. Practicalities and costs of adhesions. Colorectal Disease. 9 Suppl 2:60-5, 2007 October.

61. Fevang B T, Fevang J, Lie S A, Soreide O, Svanes K, Viste A. Long-term prognosis after operation for adhesive small bowel obstruction. Annals of Surgery. 240(2):193-201, 2004 August.

62. Binnebosel M., Rosch R., Junge K., Lynen-Jansen P., Schumpelick V., Klinge U. Macrophage and T-lymphocyte infiltrates in human peritoneal adhesions indicate a chronic inflammatory disease. World Journal of Surgery. 32(2):296-304, 2008 February.

63. Vrijland W W, Tseng L N, Eijkman H J, et al. Fewer intraperitoneal adhesions with use of hyaluronic acid-carboxymethylcellulose membrane: a randomized clinical trial. Annals of Surgery February 2002;235(2):193-199.

64. Attard J A. MacLean A R. Adhesive small bowel obstruction: epidemiology, biology and prevention. Canadian Journal of Surgery. 50(4):291-300, 2007 August.

65. Shapiro R, Young J B, Milford E L, Trotter J F, Bustami R T, Leichtman A B. Immunosuppression: evolution in practice and trends, 1993-2003. American Journal of Transplantation April 2005;5(4 Pt 2):874-886.

66. Hoffmann S C, Hale D A, Kleiner D E, et al. Functionally significant renal allograft rejection is defined by transcriptional criteria. American Journal of Transplantation March 2005;5(3):573-581.

67. Levey A S, Bosch J P, Lewis J B, Greene T, Rogers N, Roth D (1999). A more accurate method to estimate glomerular filtration rate from serum creatinine: a new prediction equation. Modification of Diet in Renal Disease Study Group (PDF). Ann. Intern. Med. 130 (6): 461-70.

68. Improvement of interobserver reproducibility of adhesion scoring systems. Adhesion Scoring Group. Fertility & Sterility. 62(5):984-8, 1994 November.

69. Rayhill S C, Heimes M, Kirk A D, Sollinger H W. Simultaneous pancreas—kidney transplantation: recent experience at the University of Wisconsin. Exp Clin Endocrinol Diabetes. 1996;104(5):353-359.

70. Gutierrez-Dalmau A., Campistol J M. The role of proliferation signal inhibitors in post-transplant malignancies. Nephrology Dialysis Transplantation. 22 Suppl 1:1-6, 2007 May.

71. Barkan H., Webster S., Ozeran S. Factors predicting the recurrence of adhesive small-bowel obstruction. American Journal of Surgery. 170(4):361-5, 1995 October.

72. Young P., Johns A., Templeman C., Witz C., Webster B., Ferland R., Diamond M P., Block K., diZerega G. Reduction of postoperative adhesions after laparoscopic gynecological surgery with Oxiplex/AP Gel: a pilot study. Fertility & Sterility. 84(5):1450-6, 2005 November.

73. Fazio V W., Cohen Z., Fleshman J W., van Goor H., Bauer J J., Wolff B G., Corman M., Beart R W Jr., Wexner S D., Becker J M., Monson J R., Kaufman H S., Beck D E., Bailey H R., Ludwig K A., Stamos M J., Darzi A., Bleday R., Dorazio R., Madoff R D., Smith L E., Gearhart S., Lillemoe K., Gohl J. Reduction in adhesive small-bowel obstruction by Seprafilm adhesion barrier after intestinal resection. Diseases of the Colon & Rectum. 49(1):1-11, 2006 January.

What is claimed is:

1. A method for preventing inflammation comprising depleting or sequestering immune lymphocytes.

2. The method of claim 1, further comprising administering to a subject one or more doses of a lymphocyte depletion agent or a lymphocyte sequestering agent or both.

3. The method of claim 1, wherein said immune lymphocytes are group consisting of B lymphocytes, T lymphocytes, NK cells, and platelets.

4. The method of claim 2, wherein said lymphocyte depletion agent is selected from the group consisting of PATG, Thymoglobulin and Alemtuzumab.

5. The method of claim 2, wherein said lymphocyte sequestering agent is FTY720.

6. The method of claim 2, wherein said lymphocyte agent is administered by a route selected from the group consisting of: oral, transdermal, transmucosal, intrademal, subcutaneous, intravenous, and intramuscular.

7. The method of claim 2, wherein said agent is administered with a pharmaceutically acceptable carrier.

8. The method of claim 1, wherein said inflammation is caused by an infection or an injury.

9. The method of claim 8, wherein said injury is ischemic injury.

10. The method of claim 12, wherein said agent is administered to the subject within 7 days before the onset of inflammation.

11. A method for treating inflammation in a subject, comprising depleting or sequestering immune lymphocytes.

12. The method of claim 11, further comprising administering to a subject one or more doses of a lymphocyte depletion agent or a lymphocyte sequestering agent or both.

13. The method of claim 11, wherein said immune lymphocytes are group consisting of B lymphocytes, T lymphocytes, NK cells, and platelets.

14. The method of claim 12, wherein said lymphocyte depletion agent is selected from the group consisting of PATG, Thymoglobulin and Alemtuzumab

15. The method of claim 12, wherein said lymphocyte sequestering agent FTY720.

16. The method of claim 12, wherein said agent is administered by a route selected from the group consisting of: oral, transdermal, transmucosal, intrademal, subcutaneous, intravenous, and intramuscular.

17. The method of claim 12, wherein said agent is administered with a pharmaceutically acceptable carrier.

18. The method of claim 1, wherein said inflammation is caused by an infection or an injury.

19. The method of claim 18, wherein said injury is ischemic injury.

20. The method of claim 12, wherein said agent is administered during or after the infection or injury.

21. The method of 19, wherein said ischemic injury is caused by hemorrhagic shock.

22. The method of 21, wherein said agent is administered before reperfusion.

23. A method for preventing abdominal adhesion, comprising depleting or sequestering immune lymphocytes.

24. The method of claim 23, further comprising administering to a subject one or more doses of a lymphocyte depletion agent or a lymphocyte sequestering agent or both.

25. The method of claim 23, wherein said immune lymphocytes are group consisting of B lymphocytes, T lymphocytes, NK cells, and platelets.

26. The method of claim 24, wherein said lymphocyte depletion agent is selected from the group consisting of PATG, Thymoglobulin and Alemtuzumab.

27. The method of claim 24, wherein said lymphocyte sequestering agent is FTY720.

28. The method of claim 24, wherein said lymphocyte agent is administered by a route selected from the group consisting of: oral, transdermal, transmucosal, intrademal, subcutaneous, intravenous, and intramuscular.

29. The method of claim 24, wherein said agent is administered with a pharmaceutically acceptable carrier.

30. The method of claim 24, wherein said abdominal adhesion is caused by an injury

31. The method of claim 30, wherein said agent is administered to the subject before, during or after the injury.


SEQUENCE LISTING (63 sequences)

SEQ ID NO: 1 - DNA, 26 bp, Artificial Sequence
  IL-1Alpha forward primer
ctgaagaagagacggttgagtttaaa

SEQ ID NO: 2 - DNA, 26 bp, Artificial Sequence
  IL-1Aplha reverse primer
aagttgtatttcatgttgctctggaa

SEQ ID NO: 3 - DNA, 31 bp, Artificial Sequence
  IL-1a probe
cagaagaagaaatcatcaagcccagatcagc

SEQ ID NO: 4 - DNA, 21 bp, Artificial Sequence
  IL-1B forward primer
ttgaattcgagtctgccctgt

SEQ ID NO: 5 - DNA, 18 bp, Artificial Sequence
  IL-1B reverse primer
cccaggaagacgggcttt

SEQ ID NO: 6 - DNA, 31 bp, Artificial Sequence
  IL-1B probe
caactggtacatcagcacctctcaagcagaa

SEQ ID NO: 7 - DNA, 21 bp, Artificial Sequence
  IL-6 forward primer
aatgtcgaggctgtgcagatt

SEQ ID NO: 8 - DNA, 21 bp, Artificial Sequence
  IL-6 reverse primer
tggtggctttgtctggattct

SEQ ID NO: 9 - DNA, 26 bp, Artificial Sequence
  IL-6 probe
agcactgatccagaccctgaggcaaa

SEQ ID NO: 10 - DNA, 17 bp, Artificial Sequence
  TNFa forward primer
tggccccttgagcatca

SEQ ID NO: 11 - DNA, 22 bp, Artificial Sequence
  TNF-a reverse primer
cgggcttatctgaggtttgaga

SEQ ID NO: 12 - DNA, 25 bp, Artificial Sequence
  TNFa probe
ccctctggcccaaggactcagatca

SEQ ID NO: 13 - DNA, 22 bp, Artificial Sequence
  EDN-1 forward primer
cctgctcttccctgatggataa

SEQ ID NO: 14 - DNA, 30 bp, Artificial Sequence
  EDN-1 probe
agtgtgtctacttctgccacctggacatca

SEQ ID NO: 15 - DNA, 23 bp, Artificial Sequence
  EDN-1 reverse primer
ggacaatgtgttctggagtgttg

SEQ ID NO: 16 - DNA, 20 bp, Artificial Sequence
  INOS forward primer
cgttatgccaccaacaatgg

SEQ ID NO: 17 - DNA, 19 bp, Artificial Sequence
  INOS reverse primer
agacccggaagtcgtgctt

SEQ ID NO: 18 - DNA, 21 bp, Artificial Sequence
  INOS probe
atcaggtcggccatcaccgtg

SEQ ID NO: 19 - DNA, 28 bp, Artificial Sequence
  HIF-1a forward primer
gcattaaagttagaaccaaatccagagt

SEQ ID NO: 20 - DNA, 23 bp, Artificial Sequence
  HIF-1a reverse primer
aaggactagctggctgatcttga

SEQ ID NO: 21 - DNA, 30 bp, Artificial Sequence
  HIF-1a probe
ctggaactttcttttaccatgccccagatt

SEQ ID NO: 22 - DNA, 22 bp, Artificial Sequence
  HSP forward primer
accatcgaggaggtggattaga

SEQ ID NO: 23 - DNA, 32 bp, Artificial Sequence
  HSP70 reverse primer
atgaagtgatacagaacagaactaaaatacag

SEQ ID NO: 24 - DNA, 31 bp, Artificial Sequence
  HSP70 probe
cctctttgagacccaaggactttgacattgt

SEQ ID NO: 25 - DNA, 21 bp, Artificial Sequence
  TGF-beta1 forward primer
agggctaccatgccaatttct

SEQ ID NO: 26 - DNA, 20 bp, Artificial Sequence
  TGF-beta1 reverse primer
cgggttgtgctggttgtaca

SEQ ID NO: 27 - DNA, 30 bp, Artificial Sequence
  TGF-beta1 probe
cctagacactcagtacagcaaggtcctggc

SEQ ID NO: 28 - DNA, 23 bp, Artificial Sequence
  C3 forward primer
caagaaatgattggtggcttcaa

SEQ ID NO: 29 - DNA, 24 bp, Artificial Sequence
  C3 reverse primer
gacctgtggttcacagatgtcttt

SEQ ID NO: 30 - DNA, 24 bp, Artificial Sequence
  C3 probe
agcctttgttctcatcgcgctgca

SEQ ID NO: 31 - DNA, 22 bp, Artificial Sequence
  TGFa forward Primer
cccagattcccacagtcagttc

SEQ ID NO: 32 - DNA, 16 bp, Artificial Sequence
  TGFa reverse primer
agcgtgcgccgacgta

SEQ ID NO: 33 - DNA, 25 bp, Artificial Sequence
  TGFa probe
aagccagcatgtgtctgccactctg

SEQ ID NO: 34 - DNA, 23 bp, Artificial Sequence
  VCAM forward primer
ctgttcaagttgctcccagggat

SEQ ID NO: 35 - DNA, 24 bp, Artificial Sequence
  VCMA reverse primer
gagcaggtcatgttcacagaactg

SEQ ID NO: 36 - DNA, 23 bp, Artificial Sequence
  VCMA probe
cgaccatctccgtcaacccctcc

SEQ ID NO: 37 - DNA, 27 bp, Artificial Sequence
  IL-2 forward primer
acagttgcttttgaaggaagttaagaa

SEQ ID NO: 38 - DNA, 22 bp, Artificial Sequence
  IL-2 reverse primer
cctgcttgggcatgtaaaattt

SEQ ID NO: 39 - DNA, 28 bp, Artificial Sequence
  IL-2 probe
cgagaatgctgatctctccaggatgctc

SEQ ID NO: 40 - DNA, 20 bp, Artificial Sequence
  CD154 forward primer
gaaatgcacaaaggcgatca

SEQ ID NO: 41 - DNA, 22 bp, Artificial Sequence
  CD154 reverse primer
tgctgttttactactggcctcg

SEQ ID NO: 42 - DNA, 28 bp, Artificial Sequence
  CD154 probe
atcctcaaattgcggcacatgtcataag

SEQ ID NO: 43 - DNA, 20 bp, Artificial Sequence
  BAX forward primer
ctcaagcgcattggagatga

SEQ ID NO: 44 - DNA, 17 bp, Artificial Sequence
  BAX reverse primer
gtccacggctgcgatca

SEQ ID NO: 45 - DNA, 28 bp, Artificial Sequence
  BAX probe
tcctctgcagctccatgttactgtccag

SEQ ID NO: 46 - DNA, 27 bp, Artificial Sequence
  BCL-2 forward primer
ttctactttgtcaggcttatgaaggtt

SEQ ID NO: 47 - DNA, 27 bp, Artificial Sequence
  BCL-2 reverse primer
tgtcctttgtcccataataattttttt

SEQ ID NO: 48 - DNA, 35 bp, Artificial Sequence
  BCL-2 probe
caggtctttattgctactcaatttgaatcttccca

SEQ ID NO: 49 - DNA, 27 bp, Artificial Sequence
  FAS forward primer
aatggtatcgaagaaaccaaaatagac

SEQ ID NO: 50 - DNA, 20 bp, Artificial Sequence
  FAS reverse primer
cggagcagctggactttctg

SEQ ID NO: 51 - DNA, 30 bp, Artificial Sequence
  FAS probe
catgcatgacaatcccaaagatacagctga

SEQ ID NO: 52 - DNA, 22 bp, Artificial Sequence
  FAS-L forward primer
ggcaagcctaactcaagatcca

SEQ ID NO: 53 - DNA, 24 bp, Artificial Sequence
  FAS-L reverse primer
catatacttcaccccagagaccaa

SEQ ID NO: 54 - DNA, 31 bp, Artificial Sequence
  FAS-L probe
caattccataggtgtcttcccattccagagg

SEQ ID NO: 55 - DNA, 20 bp, Artificial Sequence
  NF-kB forward primer
ctggcagctctcctcaaagc

SEQ ID NO: 56 - DNA, 23 bp, Artificial Sequence
  NF-kB reverse primer
cacgagtcatccaggtcatacag

SEQ ID NO: 57 - DNA, 29 bp, Artificial Sequence
  NF-kB probe
ctcaaagttctccaccaggggatctgctc

SEQ ID NO: 58 - DNA, 19 bp, Artificial Sequence
  COX-2 forward primer
ctgaacacctccgctttgc

SEQ ID NO: 59 - DNA, 25 bp, Artificial Sequence
  COX-2 RP
aagcacatcgcacactctattatgt

SEQ ID NO: 60 - DNA, 27 bp, Artificial Sequence
  COX-2 probe
cagaccaggcaccagaccaaagacctc

SEQ ID NO: 61 - DNA, 23 bp, Artificial Sequence
  PPLA-2 forward primer
ctactgtggcctaggtggatcag

SEQ ID NO: 62 - DNA, 25 bp, Artificial Sequence
  PPLA-2 reverse primer
tctctgtagcagttgtcgtgtgtct

SEQ ID NO: 63 - DNA, 25 bp, Artificial Sequence
  PPLA-2 probe
cgcagcacctgtccagttcatccac